(19) United States
(12) Patent Application Publication
(10) Pub. No.: US 2022/0273562 A1
(43) Pub. Date: Sep. 1, 2022

(54) HYDROPHOBICALLY ASSOCIATING POLYMER COUNTER ION PAIR COMPLEXES

(71) Applicant: MALVERN COSMECEUTICS LIMITED, Worcestershire (GB)

(72) Inventor: Stephen TONGE, Worcestershire (GB)

(21) Appl. No.: 17/624,771

(22) PCT Filed: Jul. 3, 2020
(86) PCT No.: PCT/GB2020/051600

§ 371 Date: Jan. 4, 2022

(30) Foreign Application Priority Data
Jul. 5, 2019 (GB) 1909680.9

(51) Int. Cl. A61K 9/107; A61K 9/08; A61K 47/18; A61K 47/34

(52) U.S. Cl. CPC A61K 9/1075; A61K 9/08; A61K 47/18; A61K 47/34

(57) ABSTRACT

There is provided inter alia a composition comprising a lipid and a hydrophobically associating charged polymer, wherein the polymer is paired with an oppositely charged counter ion bearing a hydrophobic substituent.

### SUMMARY OF INVENTION

Copolymers of maleic acid and stryene (SMA) are known to interact with phospholipids (PL) to form disc-like nanoparticles suitable for solubilizing hydrophobic or partially hydrophobic agents such as drugs or membrane proteins while maintaining the native structure of the latter. However, the only SMA copolymer that has been applied to medicine is a derivative of the 1:1 alternating copolymer, and the latter only undergoes association with PL around a collapse pH which in the case of the 1:1 SMA copolymer is around pH 4, and this is outside of the physiological range. This can be overcome by using copolymers with a higher styrene content, e.g. styrene:maleic acid (ST:MA) of 2:1 or 3:1 which interact with PL in the physiological range, but (a) the use of such copolymers limits the interaction to specific pH values of 6-8 and 7-9, respectively and (b) non-1:1 ST:MA copolymers are not readily available and are significantly more expensive than 1:1 ST:MA copolymers. To overcome this limitation the present invention has surprisingly found that the hydrophobic balance of the cheaply and widely available 1:1 SMA copolymer can be modified to react with PL over an extended pH range by making use of the effect of counter ion pairing, using a species comprising a counter ion which possess both an opposite charge to the carboxylic acid charge present in the MA repeat unit and a hydrophobic group such as a phenyl ring or short alkyl chain such as the isobutyl or isopentyl grouping attached to a amine, such as a primary amine salt. By titrating the quantity of the species comprising the counter ion, the SMA polymer chain can become associated by such ion pairing reactions with increasing quantities of hydrophobic counter ions, whereby the latter render the polymer increasingly hydrophobic until polymer collapse occurs and association with PL takes place to form disc-like macromolecular assemblies. By changing the concentration of the species comprising the counter ion, polymer collapse can occur at any given pH value over a wide pH range from pH 4-10, while only using SMA of 1:1 ST:MA. This gives great flexibility to the end user for solubilizing drugs and maintaining membrane proteins in aqueous solution for spectroscopic analysis, biochemical and binding studies. This principle can be applied more generally to all hydrophobically associating charged, amphiphilic polymers or polyelectrolytes, of both anionic and cationic type, by reaction with the appropriate and oppositely charged species comprising a counter ion to form macromolecular assemblies with PL over a wide range of pH values.

General Description

Background to Polymer/Lipid Macromolecular Assemblies:

The present invention relates to compositions for use in the solubilisation of hydrophobic substances, particularly in the solubilisation of hydrophobic active agents which are of use in the field of pharmaceuticals, and in the solubilisation of peptides and proteins for the investigation of their structure and their interactions with other substances, or for the delivery of proteins and peptides or nucleotides and oligonucleotides such as DNA and RNA to specific sites within the body, or to maintain the structure of membrane-bound proteins such that they can continue to function and can be of use in chemical and biochemical processes.

Poor water solubility presents a fundamental problem in delivering hydrophobic or oil-soluble active materials to sites within the body. Numerous formulating aids have been adopted to overcome this limitation, aiming to produce aqueous formulations that are more functionally acceptable. Approaches include the use of surfactant systems, liposomes, niosomes and cyclodextrins, amongst others. However, all of these systems have particular drawbacks. For example: liposomes and cyclodextrins may have a low loading capacity; liposomal formulations may be rapidly removed from the systemic circulation after intravenous administration; both liposomes and niosomes may suffer from a lack of optical clarity and high particle size >100 nm diameter; and the use of surfactants may result in the formation of toxic or irritant compositions.

Many potent drugs are hydrophobic in nature and show poor water solubility and present a challenge to formulate into aqueous formulations suitable for intravenous drug delivery, this is often the result of structures developed to interact with active hydrophobic pharmacophores within target proteins that by necessity require active molecules to have a partially hydrophobic nature that in turn renders them water insoluble. There is a need for a vehicle which can contain and deliver such agents to the active site while maintained in aqueous solution.

Hydrophobically associating charged polymers (also known as amphipols or amphiphilic polymers, due to their amphiphilic character, hypercoiling polymers or hypercoiling polyelectrolytes) may associate with phospholipids to form flattened disk-like macromolecular assemblies.

Hydrophobically associating polymers are known in the art. Further definition of, and information on hydrophobically associating polymers may be found in Tonge, S R and Tighe, B J. Responsive hydrophobically associating polymers: A review of structure and properties¹.

A charged polymer is considered ‘hydrophobically associating’ if the charged polymer contains hydrophobic groups, suitably incorporated into the polymer usually as pendant groups to the polymer backbone. The presence of such hydrophobic groups result in the charged polymer forming a three-dimensional structure on exposure to water in which the hydrophobic and hydrophilic groups are spatially separated into two distinct domains presenting two facets, this is particularly the case upon neutralisation as the charged groups lose their charge leading to a loss of mutual repulsion and chain collapse.

Suitably the hydrophobically associating charged polymer is selected from a polymer having a carbon backbone (i.e. the longest series of covalently bonded atoms that together create the continuous chain of the polymer molecule are carbon), a polycarbonate, a polyester, a polyether, a polyphosphate, a polyurea or a polyurethane. More suitably the hydrophobically associating charged polymer is selected from a polymer having a carbon backbone or a polyester. More suitably the hydrophobically associating charged polymer is a polymer having a carbon backbone.

Alternatively, or suitably in addition, the hydrophobically associating charged polymer does not comprise an amide linkage and/or does not comprise a peptide bond.

For example, homopolymers of ethacrylic acid (i.e. poly[2-ethacrylic acid], also known as PEAA) have been shown to interact with pure DLPC, DMPC, DPPC, DSPC (respectively di-lauryl, di-myristyl, di-palmityl and di-stearyl phosphatidyl choline) and DPPG (di-palmityl phosphatidyl glycerol), and also a mixture of DPPC/DPPA (di-palmityl phospatidic acid) resulting in the formation of optically clear, aqueous solutions^{2,3,4}. This effect is the result of a conformational transition from the extended chain, typical of a polyelectrolyte, through an intermediate state as a random coil, to a compact hypercoiled state at low pH, below the apparent pKₐ of the poly acid in question. In the partially charged or intermediate state the polyacid possesses both hydrophilic and hydrophobic characteristics within the same polymer molecule.

Other hydrophobically associating charged polymers are also known to interact with PLs to form macromolecular assemblies, such as copolymers which contain hydrophilic and hydrophobic monomer components. International Patent Application WO99/009955 (equivalent to granted patents EP1007002 and U.S. Pat. No. 6,436,905)⁵ discloses the use of hydrolysed alternating copolymers of maleic anhydride (anionic, hydrophilic in its hydrolysed maleic acid form) and either styrene or an alkyl vinyl ether (hydrophobic). Particulate structures in the region of 10-40 nm in diameter were prepared using a hydrolysed 1:1 alternating polymer of styrene and maleic anhydride (hydrolysed to maleic acid), in conjunction with pure DLPC or DPPC as detailed by Tonge and Tighe¹).
1. Tonge, S R and Tighe, B J. Responsive hydrophobically associating polymers: A review of structure and properties. Advanced Drug Delivery Reviews. 53:109-122 (2001).
2. Seki, K and Tirrell, D Macromolecules 17:1692-1698 (1983).
3. Tirrell, D, Takigawa, D and Seki, K. Ann. New York Acad. Sci. 446:237-248 (1985).
4. Thomas, J L, Devlin B P and Tirrell, D A Biochimica et Biophysica Acta. 1278:73-78 (1996).
5. Tonge, S. R. & Tighe, B. J. Lipid-containing compositions and uses thereof, U.S. Pat. No. 6,436,905. Aug. 20, 2002, to Aston University (2002).

### Alternating Copolymers of Styrene and Maleic Anhydride

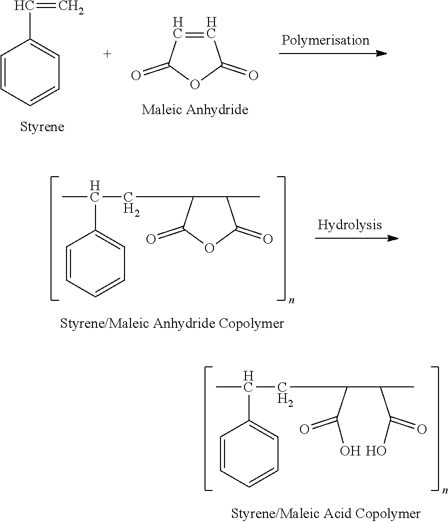

The surface activity of hydrophobic polycarboxylates such as PEAA and SMA (i.e. hydrolysed styrene/maleic anhydride polymer) is dependent upon the polymer shape and the protonation states of the pendant carboxylic acid groups. Potentiometric titration of SMA copolymers indicates that when around 50-20% of the primary (α₁) carboxylic acid groups of maleic acid (MA) remain ionised, an increase in apparent pKₐ is observed as the polyanion starts to behave as a weaker acid, indicating a conformational change in the polymer. This transition to a more compact or hypercoiled polymer structure occurs over the so-called ‘collapse pH range’ when between 100%-20% of the α₁ groups remain ionised and all of the secondary (α₂) groups of MA are unionized⁶.

In general terms, the maximum surface activity of 1:1 SMA polymers occurs when 50-20% of the α₁ carboxylic acid groups are ionised⁷ and corresponds to the lower end of the collapse pH range (potentiometric)⁶ and it is under these conditions that interaction with PL occurs to form disc-like macromolecular assemblies (SMA/PL).
6. Ohno, N., Nitta, K., Makino, S. & Sugai, S. Conformational Transition of the Copolymer of Maleic Acid and Styrene in Aqueous Solution. J Polym Sci Part A-2 Polym Phys 11, 413-425 (1973).
7. Boiko, V. P. Surface-tension of aqueous-solutions of a copolymer of styrene and maleic acid at interface with air. Colloid Journal of the USSR 38, 486-489 (1976).

SMA/PL macromolecular assemblies have potential for drug delivery purposes, as a consequence of their stability upon dilution (unlike conventional micelles or bicelles) while their nano-molecular dimensions make them ideal candidates for cellular and sub-cellular delivery and particularly suited for interaction with sub-cellular organelles.

Such polymer/PL macromolecular assemblies have been proposed as a means for the solubilisation of active agents with poor aqueous solubility. However, both PEAA and SMA phospholipid systems suffer from a number of disadvantages. PEAA is not commercially available and its suitability for use in pharmaceuticals has not yet been determined. Furthermore, these synthetic polymers only interact with phospholipids to form macromolecular assemblies at a pH level near to or below their respective pKₐ value, in the case of PEAA this is 6.5^{9, 10}.
9. Fichtner, F & Schonert, H Colloid & Polymer Sci. 255:230-232 (1977);
10. Thomas, J L, Devlin B P & Tirrell D A. Biochimica et Biophysica Acta. 1278, 73-78 (1996).

While alternating 1:1 copolymers of styrene and maleic acid (i.e. hydrolysed styrene/maleic anhydride polymers) have a pKₐ value in the region of 3.75-4.0¹¹, the pKₐ for the individual acid functions being approximately 1.97 and 6.24. Preparation of clear solutions, and hence macromolecular assemblies with PL, requires a lowering of the pH to between 3-5. Such pH levels are not generally suitable for compositions which are to be applied to mucosal surfaces of the body or suitable for use as an injectable medication or for the maintenance of higher order structure within proteins. Although the pH of these alternating copolymer formulations may be raised after the formation of the polymer/lipid macromolecular assemblies, such adjustment leads to instability, which may be observed as a loss of optical clarity over time as the macromolecular assemblies dissociate.

The styrene/maleic anhydride and in particular the corresponding maleic acid hydrolysis product and half esters have been employed widely in industrial and household applications, including use as coatings, sizing agents and for emulsification and dispersant purposes, these polymers have received limited application in biomedical products.
11. Sugai, S and Ohno, N. Conformational transitions of the hydrophobic polyacids. Biophys. Chem. 11, 387-395 (1980).

Indeed, the alternating or 1:1 copolymer of maleic acid and styrene1:1 (SMA) is the only polymer of the hydrophobically associating family so far used for producing polymer/PL macromolecular assemblies where the polymer in question has also been used as part of an injectable medicament in clinical studies in the form of a butyl half ester known as SMANCS¹², as described in U.S. Pat. No. 4,732,933, which degrades in vivo into SMA and has been marketed as a pharmaceutical product.
12. Maeda, H. SMANCS and polymer-conjugated macromolecular drugs: Advantages in cancer chemotherapy. Adv. Drug Deliv. Rev. 46, 169-185 (2001).

To date only 1:1 SMA polymer could be directly applied to drug delivery in man, without the need for further extensive preclinical toxicology studies. However as described this polymer does not form macromolecular assemblies with PL in the physiological pH range. While 2:1 and 3:1 (S:MA) copolymers do react with PL to form nanoparticles in the physiological pH range they have not been applied clinically. There is therefore and overwhelming need to render 1:1 SMA copolymer reactive with PL at physiological pH such that the resultant macromolecular assemblies can be used both to deliver drugs at physiological pH and can also carry proteins for the purpose of membrane solubilisation and hence characterisation by spectrophotometric and other biochemical means. There is also a need to make such SMA/PL macromolecular assemblies and related polyanion/PL macromolecular assemblies tolerant to divalent cations. In addition the removal of pendant aromatic hydrophobic groups from the polymer chain and replacement with non-aromatic groups would prevent UV absorption that would otherwise occur in the presence of styrene repeat units and therefore interfere with spectroscopic measurements that depend upon such absorbance e.g. UV absorption studies.

The hydrophobically associating charged polymer may comprise either positive or negative charges, resulting in a net positive or negative charge. A mixture of hydrophobically associating charged polymers may be included in the composition of the invention, wherein the hydrophobically associating charged polymers may have the same, or alternatively, a mixture of different charges. For example, the hydrophobically associating charged polymer may comprise a mixture of a copolymer of dimethylaminopropylamine maleimide and styrene (SMI) or copolymers of other maleimide derivatives and stryrene or a terpolymer of maleic acid and styrene and dimethylaminopropylamine maleimide. Alternatively, in particular embodiments the hydrophobically associating charged polymer comprises only negative charges (i.e. does not comprise positive charges) or comprises only positive charges (i.e. does not comprise negative charges).

### Background to Ion Pairing in Polymers

Although it is well known that polyelectrolytes associate with ions of opposite polarity and this results in a collapse of the polymer chain and reduction in polymer dimensions such as mean end-to-end polymer distance or radius of gyration, as described by Winkler (1998)¹³, Schiessel (1998)¹⁴, Kundagrami (2008)¹⁵ and Roiter (2010)¹⁶ (and this ion-pairing induced collapse may be irreversible Wei (2012)¹⁷), in such cases the ion pair consist of two oppositely charged ions so close to each other that other ions cannot screen their interaction and they appear to behave in a manner similar to single undissociated molecules.
13. Winkler, R. G., Gold, M. & Reineker, P. Collapse of Polyelectrolyte Macromolecules by Counterion Condensation and Ion Pair Formation: A Molecular Dynamics Simulation Study.
   Phys. Rev. Lett., 80, 3731 (1998).
14. Schiessel, H, & Pincus, P.
   Counterion-Condensation-Induced Collapse of Highly Charged Polyelectrolytes.
   Macromolecules, 1998, 31 (22), 7953-7959.
15. Kundagrami, A. & Muthukumara, M. Theory of competitive counterion adsorption on flexible polyelectrolytes: Divalent salts. J. Chem. Phys. 2008, 128, 244901 (1-16).
16. Roiter, Y., Trotsenko, O., Tokarev, V.^{†}, & Minko, S. Single Molecule Experiments Visualizing Adsorbed Polyelectrolyte Molecules in the Full Range of Mono- and Divalent Counterion Concentrations. J. Am. Chem. Soc., 132 (39), 13660-13662 (2010).
17. Wei, X. & Ngai, T. Ion-induced hydrophobic collapse of surface-confined polyelectrolyte brushes measured by total internal reflection microscopy. Polymer Chem. 8, (2012).

Although Muñoz-Guerra describes ionic complexes between natural poly(beta, L-malate) (PMLA) and alkyltrimethylammonium sulphates^{18,19} as a method of containing erythromycin within a solid degradable polymer particle for slow surface erosion, there is no suggestion that these complexes could be reacted with PL to form water soluble or macromolecular assemblies suitable for drug deliveryl⁹. Indeed these authors describe a stoichiometric or nearly stoichiometric interaction between the counter ion and all of the polymeric pendant carboxylic acid groups to render the complex less water soluble, such complexes are solid crystallisable materials that exhibit melting at temperatures between 40 and 70° C.²⁰. Similar ionic association complexes are described for polypeptides such as poly(gamma, D-glutamate) anions and alkyltrimethylammonium cations²¹ formed by precipitation from aqueous solution to form water insoluble, layered paraffinic solids some of which were crystallisable depending upon the length of the alkyl chain.
18. Garcia-Alvarez M, Martinez de Ilarduya A, Portilla J A, Alla A, Muñoz-Guerra S. Ionic complexes of biotechnological polyacids with cationic surfactant. Macomol. Symp., 273, 85-94 (2008).
19. Portilla-Arias J A, Garcia-Alvarez M, Martinez de Ilarduya A, Muñoz-Guerra S.
   Ionic complexes of biosynthetic poly(malic acid) and poly(glutamic acid) as prospective drug-delivery systems. Macromol. Biosci., 7, 897-906 (2007).
20. Portilla-Arias J A, Garcia-Alvarez M, Martinez de Ilarduya A, Holler E, Muñoz-Guerra S.
   Nanostructurated complexes of poly(beta, L-malate) and cationic surfactants: synthesis, characterization and structural aspects. Biomacromolecules, 7(1), 161-70 (2006).
21. Pérez-Camero G, Garcia-Alvarez M, Martinez De Ilarduya A, Fernández C, Campos L, Muñoz-Guerra S. Comblike complexes of bacterial poly(gamma,d-glutamic acid) and cationic surfactants. Biomacromolecules, 5(1):144-52 (2004).

From the foregoing examples it is not obvious that chain collapse or complexation can be controlled in such a manner that distinct hydrophobic domains could be formed to enable interaction between a polyelectrolyte such as a poly(carboxylic acid) e.g. SMA 1:1 and PL, to form a macromolecular assembly at physiological pH values, analogous in structure to those described with non-alternating SMA e.g. 2:1 and 3:1 S:MA ratios at physiological pH values of 7-8. (Tonge, U.S. Pat. No. 8,623,414)²².
22. Tonge, S R. Compositions comprising a lipid and copolymer of styrene and maleic acid. U.S. Pat. No. 8,623,414. Jan. 7, 2014 to Malvern Cosmeceutics Limited.

In order to overcome these limitations and extend the versatility of hydrophobically associating charged polymers, including both anionic and cationic polymers, to form polymer/PL macromolecular assemblies we describe herein the novel use of ion pairing to combine said polyelectrolytes with ion pairs that have both an opposite charge to the charge on the polymer chain together with hydrophobic moieties in the same molecule, for example, a typical ion pair will be the hydrochloride salt of phenylethylamine (PEA) which in its salt form will possess a quaternary nitrogen. The charged PEA can be introduced to 1:1 SMA preferably in aqueous solution such that the positively charged nitrogen groups ion pair with the negatively charged carboxylic acid groups of the styrene maleic acid copolymer (SMA) in such a manner as to raise the apparent pKₐ of the SMA and move the collapse pKₐ from approx. pH 4-5 to pH 8-9 (See results in Table 1). This collapse pH can be modified by titrating with specific amounts of PEA solution so as to produce a specific collapse pH in a pH region from 4-10 and therefore control the range over which association with PL and formation of polymer/PL macromolecular assemblies occur.

This invention offers a distinct advantage for the user in that a single grade of SMA 1:1 polymer, which is the most widely available commercial grade of SMA, and also the cheapest grade of such material, can be adjusted to collapse over a wide range of pH values and can therefore be used to suit a range of drugs, or proteins or oligonucleotides that are to be contained within or upon the SMA/PL macromolecular assemblies so formed. The additional advantage of using a species comprising a counter ion such as PEA is that such materials are widely used as nutritional supplements with a known safety profile and the associated polymer ion pairs are not considered as new chemical entities (NCEs).

By reacting pendant anionic groups of 1:1 SMA polymer with cationic groups of the ion pair the net charge on the polymer can be rendered amphoteric or neutral allowing intra-chain charge repulsion to be partially or fully removed and chain collapse to occur depending upon the amount of ion pair present, the latter can be readily titrated by those skilled in the art. In particular embodiments, the amphoteric nature of the resultant ion-pair complex described herein renders the resultant polymer/PL macromolecular assembly insensitive to cationic charges such as Ca²⁺ or Mg²⁺ ions or salts and thereby overcome some of the drawbacks associated with using SMA e.g. for characterising membrane proteins that require the presence of these ions for their correct function.

By combining the 1:1 SMA with a variety of single charged hydrophobic cations a range of ion pair complexes have been produced with differing charge densities at differing pHs depending upon the nature of the charge moiety involved and their respective pK values, e.g. if imidazoline is used in place of a primary amine, as is the case for 2-benzyl-2-imidazoline (tolazoline) the pK of the imidazoline group behaves as a weaker base and therefore the collapse pK of the 1:1 SMA polymer, when associating with PL, is lowered to approx. pH 7. In this way the range of pHs over which 1:1 SMA can operate to form polymer/PL macromolecular assemblies is broadened. By use of straight chain primary amines such as isobutyl or isopentyl amine the aromatic groups present in PEA or 2-benzyl-2-imidazoline can be avoided. By combining such aliphatic amine salts with poly carboxylic acids that also lack aromatic pendant groups such as styrene, e.g. with poly(malic acid) or poly(beta-malic acid) or poly(maleic acid), the resulting polymer/PL macromolecular assembly will not absorb UV light and will not therefore interfere with UV spectroscopic analysis of proteins or actives contained in such polymer/PL macromolecular assemblies.

The scope of suitable species comprising counter ions that can be used can be greatly expanded to include many drugs directly as counter ions or species comprising counter ions since many drugs are the salts of weak bases, especially those acting on the adrenergic or sympathetic nervous system, and often have an aromatic ring as part of their structure as is the case for catecholamines, or sympathetic neurotransmitters and their respective agonist and antagonist drugs. Some of these agents can also surprisingly be used as ion pairs for the application described herein in their charged state as hydrochloride salts, e.g. diphenhydramine, tyramine, naphazoline, in addition amino acids and their C-substituted derivatives can also be used as ion pairs such as L-phenylalanine ethyl ester hydrochloride (See Structures That Form Working Ion Pairs). The polymer/PL macromolecular assemblies formed between polymers and PL and the amine drugs listed (See results in Table 1) could be used as a delivery system for the drugs involved enabling them to be delivered across biological membranes such as the dermal-blood barrier, gut-blood barrier or blood-brain barrier to achieve preferential entry into selected compartments within the body.

There seems to be an optimal counter ion size, where bulky aromatic groups prevent polymer chain collapse, while a two carbon spacer unit (C2H4) is present between the charged amine and aromatic ring structure, without disubstitution of the attached aromatic ring by either hydroxyl, methyl or methoxy groups.

Suitably, the species comprising a polymer large enough to present separately its charged hydrophilic and hydrophobic regions.

Suitably the species comprising a polymer has a molecular weight of no greater than 100 kDa, more suitably no greater than 50 kDa, more suitably no greater than 25 kDa, more suitably no greater than 15 kDa, more suitably no greater than 10 kDa.

Suitably the species comprising a polymer has a molecular weight of 1 kDa to 20 kDa, more suitably 2 kDa to 10 kDa, more suitably 3 kDa to 9 kDa, more suitably 5 kDa to 7 kDa, more suitably about 6 kDa.

Suitably the counter ion is divalent or monovalent. More suitably, the counter ion is monovalent.

The following species comprising cationic counter ions failed to ion pair with SMA 1:1 in a manner which led to formation of SMA/PL macromolecular assemblies; hydrochloride salts of benzocaine, 4-aminophenol, normetanephrine, 4-amino benzoic acid (PABA), L-norepinephrine, aniline, lidocaine, triethanolamine, dopamine, octopamine, L-dopa, while amitryptyline hydrochloride only partially formed such SMA/PL macromolecular assemblies (See Structures That Form Partial Working Ion Pairs & Structures That Fail to Form Working Ion Pairs).

The additional advantage of using approved drug molecules as species comprising a counter ion for ion pairing purposes is that they have a known toxicological profile and the resulting polymer-counter ion pairs are not considered as new chemical entities (NCEs).

In a further embodiment of the invention, the charged polymer could be a straight chain polyelectrolyte, such as a polyanion, or alternatively, a polycation, without covalently bonded pendant hydrophobic groups e.g. aromatic or alkyl or alkenyl chains, and therefore not prone to hydrophobic association, but by counter ion pairing a hydrophobically associating polymer could be formed in situ by mixing with a counter ion bearing an opposite charge to that of the polyelectrolyte but linked to an appropriate hydrophobic group, such as a phenyl or benzyl group and by selection of such an appropriate counter ion the resulting polyelectrolyte counter ion pair will behave as a hydrophobically associating polymer and will interact with PL to form polymer counter ion/PL macromolecular assemblies. Further, by selection of a suitable concentration of counter ion salt the formation of such macromolecular assemblies can be made to occur over a wide range of pH values including over the physiological range.

Example polyanion-ion pairs include; poly(acrylic acid), poly(crotonic acid), poly(methacrylic acid) or poly(maleic acid), combined with ion pairs bearing a cationic charge such as a primary amine covalently linked with either an aromatic group such as PEA or an alkyl group such a isobutyl or isopentyl amine, or alternatively, an imidazoline group such as 2-benzyl-2-imidazoline (tolazoline) covalently linked to an aromatic group.

In a further embodiment of the invention the polymer could be a polyanion with a biodegradable backbone such as a polyester, examples of ester polymers include poly(malic acid) or poly(beta-malic acid). In these cases suitable amine or nitrogen containing cation pairs include PEA, isobutyl and isopentyl amine or tolazoline. These degradable polymer cation pairs and resulting macromolecular assemblies formed with PL are particularly useful for drug delivery purposes and for use as injectable pharmaceuticals since they will biodegrade into components that are found as part of normal metabolic processes or as dietary components and will therefore be harmlessly metabolised or excreted from the body after introduction to the body by absorption or injection.

Ideally, the net charge of the hydrophobically associating charged polymer is particularly countered by the species comprising a counter ion. Suitably, sufficient charge remains ‘uncountered’ on the charged polymer to prevent the polymer from ‘salting out’ of solution (i.e. the polymer remains in solution). Accordingly, in one embodiment, the concentration of species comprising a counter ion in the composition is such that the hydrophobically associating charged polymer is in solution.

The polymer and lipid should form a macromolecular assembly due to the inclusion of the counter ion in the composition of the invention. Accordingly, in one embodiment there is provided a composition comprising a lipid, a hydrophobically associating charged polymer and a species comprising a counter ion, wherein the counter ion is oppositely charged to the polymer, wherein the polymer and lipid are in the form of a macromolecular assembly and wherein, in the absence of the counter ion, the polymer and lipid would not be in the form of a macromolecular assembly.

Applications:

Example 1, Changing Polymer Behaviour by Increasing Counter Ion Concentration: By varying the concentration of the species comprising a counter ion the pH at which the SMA polymer chain collapses into hydrophobic domains can be titrated, e.g. by combining SMA 1:1 with increasing concentrations of PEA the pH over which the polymer collapses to interact with phospholipid increases from 4.5 to 7.0 to 8.5 as the ratio of SMA(1:1):PEA is raised from 1:0 to 9:1 to 5:4 (See results in Table 1).

Example 2, Counteracting Incompatibility with Divalent Cations: By using a species comprising a cationic counter ion such as PEA in combination with an anionic polymer such as SMA 1:1 and forming an initial complex, the subsequent ability of the polyanion to bind with other cations such as divalent cations e.g. Ca²⁺ and Mg²⁺ is mitigated, this can be a great advantage when solubilizing proteins within a SMA/PL macromolecular assembly which require divalent cations to function. In comparison, the use of SMA 2:1 or 3:1 to form the SMA-PL particle would generally result in precipitation with M²⁺ ion concentrations above 1 mM²³, limiting the use of the existing SMA-based solubilisation systems which cannot be used to solubilise membrane proteins that require high concentrations of divalent ions, examples include proteins that bind and/or hydrolyse ATP such as ABC transporters that are typically purified in buffers containing a minimum concentration of 50 mM Mg²⁺ or in some ion channels, where divalent cations can promote oligomerization and ensure functionality²⁴.
23. Esmaili, M. & Overduin, M. Membrane biology visualized in nanometer-sized discs formed by styrene maleic acid polymers. Biochim. Biophys. Acta—Biomembr. 1860, 257-263 (2018).
24. Alexander, S. P. H., Mathie, A. & Peters, J. A. Guide to Receptors and Channels. British Journal of Pharmacology, 164, Suppl 1:S1-324. (2011).

Example 3, Use of Alternative Polyanions: A hydrolysed copolymer of diisobutylene and maleic anhydride (DIBMA) has also been shown to form polymer/PL macromolecular assemblies (12 nm to 30 nm diameter), while DIBMA shows improved salt tolerance to high concentrations of divalent cations, and more importantly, lacks styrene groups, which in SMA result in absorbance in the UV spectral range, thus enabling DIBMA to be used for circular dichroism and other forms of absorption spectroscopy operating in the UV range^{25,26,27}, However the pH at which DIBMA reacts with PL is fixed and the use of cation pairing, by using non-aromatic salts of aliphatic amines, enables this pH to be extended to a much wider range than currently available by using DIBMA alone.
25. Oluwole, A. O. et al. Formation of Lipid-Bilayer Nanodiscs by Diisobutylene/Maleic Acid (DIBMA) Copolymer. Langmuir, 33, 14378-14388).
26. Barniol-Xicota, M. & Verhelst, S. H. L. Stable and Functional Rhomboid Proteases in Lipid Nanodiscs by Using Diisobutylene/Maleic Acid Copolymers. J. Am. Chem. Soc. 7, 140(44), 14557-14561(2018).
27. Oluwole, A. O. et al. Solubilization of Membrane Proteins into Functional Lipid-Bilayer Nanodiscs Using a Diisobutylene/Maleic Acid Copolymer. Angew. Chemie—Int. Ed. 56, 1919-1924 2017)

Alternatively a homopolymer of itaconic acid could be used in combination with cationic counter ions in the form of aromatic or aliphatic amine salts, such polymers are sold under the brand name Amaze™ SP Polymer in its sodium salt form by Nouryon and are of low molecular weight

Example 4, Direct Solubilization of Biological Membranes: SMA 1:1 in combination with closely bonded cationic counter ions such as PEA can also be used to directly solubilise cellular membranes either from whole cells or from homogenised cellular fractions containing segments of intact membrane sourced from either prokaryotic or eukaryotic cells as a method of directly solubilising endogenous membrane bound proteins from cell membranes or as a means of sampling endogenous lipid composition as part of lipidomic studies without the requirement for additional exogenous PL. Or alternatively, as a means of solubilizing specific membrane components to expose parts of the cell, for extractions of agents contained within those sub-cellular compartments or organelles, e.g. to selectively remove the outer membrane of a Gram negative bacterium such as E.Coli to expose the periplasmic space and thereby release components from that space such as proteins that may otherwise be difficult to extract without disrupting the whole cell and having to overcome problems associated with separation of individual components from whole cells. This methodology enables use of readily and cheaply available SMA 1:1 copolymer rather than less available and more expensive block copolymers such as SMA 2:1 and 3:1. The instant invention can therefore be used as a flexible processing aid for bioprocessing and protein extraction. Or additionally, to sample lipid components from living cells in lipidomics studies28.
28. Bada Juarez J F, O'Rourke D, Judge P J, Liu L C, Hodgkin J & Watts A. Lipodisqs for eukaryote lipidomics with retention of viability: Sensitivity and resistance to Leucobacter infection linked to C.elegans cuticle composition. Chem Phys Lipids. August 222, 51-58 (2019). Epub 2019 May 15.

Example 5, Membrane Protein Extraction and Characterisation: SMA 1:1 in combination with closely bonded cationic counter ions such as PEA and PL can also be used to directly extract membrane proteins. This technique has a distinct of advantages over conventional methods of membrane protein solubilisation such as bicelle, Saposin-nanodisc or MSP-nanodisc formation, in that an initial step of detergent solubilisation is not required thereby minimising disruption of lipid-protein interactions. In addition, both faces of the embedded membrane protein remain accessible to ligands and substrates, while the bilayer structure of the disc reproduces the lateral pressure profile of native membrane more accurately than detergent micelles²⁹.
29. Bada Juarez J F, Harper A J, Judge P J, Tonge S R, & Watts A. From polymer chemistry to structural biology: The development of SMA and related amphipathic polymers for membrane protein extraction and solubilisation. Chem Phys Lipids. 221, 167-175 (2019). Epub 2019 Mar. 30.

Example 6, Advantages over Existing Copolymers for Solubilising Membrane Proteins: SMA copolymers have a number of disadvantages when solubilising membrane proteins, these have been overcome by the use of specific alternative polymers, some of which need to be specifically synthesised for this purpose, are expensive, not readily available and have no history of clinical application or use and may confer considerable toxicity. The present invention allows these disadvantages to be overcome and avoid the requirement for specific polymer types to overcome problems associated with the concurrent use of divalent cations, use at low or high pH environments or for the absence of aromatic e.g. styrene groups, within the polymer chain to avoid UV absorption by such groups and interference with UV absorption studies.

While a hydrolysed copolymer of diisobutylene and maleic anhydride (DIBMA) has also been shown to form polymer/PL macromolecular assemblies (12 nm to 30 nm diameter), and exhibits improved salt tolerance to high concentrations of divalent cations either by coulombic screening (charge shielding) or partial neutralisation of the polymer carboxylate groups³⁰.
30. Danielczak B, Meister A, & Keller S Influence of Mg2+ and Ca2+ on nanodisc formation by diisobutylene/maleic acid (DIBMA) copolymer. Chem Phys Lipids. 221:30-38 (2019).

In one embodiment, the hydrophobically associating charged polymer is not DIBMA and/or the species comprising a counter ion is not Ca²⁺ or Mg²⁺ ions.

Example 7, Use of Amine Drugs as Species Comprising a Counter Ion: A wide range of cations can be used as counter ions including many drugs which may themselves consist of species that contain counter ions, therefore such drugs can be directly associated with the polyanion chain, polyanions listed; including SMA 1:1, SMA 2:1 or 3:1 or 4:1 or DIBMA, can be used in combination with aromatic amine drugs such as those listed in Table 1, typically catecholamines are most suitable, such examples most often act as agonists and antagonists in the mammalian sympathetic nervous system, acting on adrenergic receptors or adrenoceptors, such polyanion drug counter ion combinations can then be associated with PL to form macromolecular assemblies whereby the bound drug constitutes part of the hydrophobic moiety of the hydrophobically associating polymer thereby forming a direct nanostructured delivery system for the bound drug.

These ionically associating polymer-drug conjugates are distinct from polymer-drug conjugates where the drug is usually covalently bonded to the polymer via a biodegradable bond, e.g. typically a hydrolysable ester or amide bond. The latter polymer-drug conjugates require chemical reaction to form so changing the drug structure and constituting a novel material which could not be readily applied clinically without costly toxicological studies. In contrast, the structures proposed herein are formed by reversible ionic interactions that do not modify the chemical structure of the starting components including the drug molecules.

Example 8, Use of Straight Chain Polyions Without Hydrophobic Pendant Groups: Polyanions or polycations without covalently bonded pendant hydrophobic side chains, e.g. the polyanion, poly(maleic acid) can also be combined with a hydrophobic species comprising counter ions such as PEA at concentrations that render the polymer backbone susceptible to hydrophobic association and collapse as the charge is removed upon neutralisation of the carboxylic acid groups forming a hydrophobically associating polymer in situ. The resulting hydrophobically associating polymer ion pair can interact with PL to form polymer/PL macromolecular assemblies.

Example 9, Use of Biodegradable Polyelectrolytes: Polyanions or polycations with a biodegradable backbone can also be used in another embodiment of the instant invention, preferably a polyester, subject to breakdown by enzymatic hydrolysis in the body to release components that are part of normal metabolism and ultimately either harmlessly excreted or metabolised to carbon dioxide and water. Examples include poly(malic acid) or poly(beta-malic acid).

Example 10, Use of Polycations 1: Additional suitable polycations include the copolymer of styrene and dimethylaminopropylamine maleimide (SMI), whether alternating (1:1) or block copolymers such as styrene:maleimide 3:1 and 2:1, modified by ion pairing with hydrophobic anion species comprising counter ions such as benzoic acid in its sodium or potassium salt form to change the pH at which the SMI-counter ion pair associates with PL to form polymer counter ion/PL macromolecular assemblies suitable for carrying membrane proteins, or polypeptides, or nucleotides, or oligonucleotides for characterisation or delivery purposes e.g. drug delivery.

As a further embodiment, polycations can also be rendered hydrophobic by ion pairing with hydrophobic anions including aromatic counter ions such as phenyl propionic acid- or trans-cinnamic acid, or phenyl lactic acid in their potassium and sodium salt forms, or with aliphatic counter ions such as sorbic acid or propionic acid in their potassium and sodium salt forms.

Example 11, Use of Polycations 2: Poly(itaconic acid) and its associated zinc salts can be used as a polycation for ion pairing with suitable aromatic or aliphatic anion species comprising counter ions to render the polycation hydrophobic. The association product of poly(itaconic acid) and zinc known commercially as Zinor behaves as a polycation and interacts with benzoic acid sodium salt (see Table 1), the resulting hydrophobically associating polymer can associate with PL to form polymer counter ion/PL macromolecular assemblies suitable for carrying membrane proteins, or polypeptides, or nucleotides, or oligonucleotides for characterisation or delivery purposes e.g. drug delivery.

Example 12, Formation of High Surface Area Solutions: Any one of examples 1 through 9 could be used to produce solutions containing high concentrations of macromolecular assemblies and their associated bilayer membranes, where the total surface area of membrane is extremely high, whereby a single 1 L solution containing 10% w/w aqueous solution of the polymer/PL macromolecular assemblies described would possess a total surface area comprising thousands of square meters making them ideally suited as a surface for conducting catalytic reactions that occur only at interfaces, e.g. enzyme catalysed reactions such as those involved in photosynthesis or photobiology for use in energy generation from solar power or for carbon capture where a large interfacial area contained within a small volume is essential for processing efficiency.

More generally, there is clearly a need to produce a stable, non-irritating formulating aid that enables oil-soluble active agents to be incorporated into an aqueous medium at high concentration, while at the same time forming macromolecular assemblies that are small enough to be absorbed by cells.

According to the present invention there is also provided a composition comprising a lipid and copolymer of styrene and maleic acid, wherein the ratio of styrene to maleic acid monomer units is 1:1 combined with a cationic counter ion, wherein the polymer cationic counter ion pair and lipid are in the form of macromolecular assemblies. Such compositions are examples of compositions of the invention.

Monomer ratios stated for polymers are defined on the basis of the number of each monomer unit in the polymer, for example, a ratio of styrene and maleic anhydride of 1:1 indicates that there is one styrene monomer unit for each maleic anhydride monomer unit in the polymer chain. It will be understood that the stated monomer ratios are averages and, as a result of the uncertainty in polymerisation reactions, do not necessarily represent the exact ratio for any specific polymer chain. Typically 50%, suitably all of the polymer chains will have a monomer ratio which is within 25% (for example within 15%), more particularly within 10% and especially within 5% of the stated value. For example, a ratio of styrene and maleic anhydride of 1:1 may also be described as being 50% styrene, 10% variation of this range covers copolymers having a styrene content from 45%-55%. Copolymers having a monomer ratio of 1:1 may be alternating or may be blocky in nature, depending upon the monomers present and the process of manufacture. Suitably, the hydrolysed styrene/maleic anhydride copolymer of use in the present invention will be alternating, i.e. the styrene and maleic acid residues will be arranged in an alternating relationship.

Styrene/maleic anhydride copolymers are conveniently prepared by a precipitation process, typically in an aromatic hydrocarbon solvent, for example toluene or dichlorobenzene. Polymerisation may be initiated using free-radical initiators, for example AIBN (azoisobutyronitrile) and the molecular weight may be controlled by the use of end-capping agents such as alkylated aromatic hydrocarbons, for example p-cymene. The ratio of monomers in the polymer may be controlled by variation of the monomer feed composition, and may be determined by means known to those skilled in the art, for example by titration to determine maleic acid content of the hydrolysed polymer.

The presence of a macromolecular assembly (an association of individual molecules within a macromolecular structure which is not maintained by covalent bonding), also referred to as a macromolecular complex, may be confirmed by a number of means available to those skilled in the art for the determination of particle size, for example, electron microscopy (such as used by Tonge and Tighe¹, for macromolecular assemblies incorporating alternating styrene/maleic acid copolymers) or laser diffraction techniques. However, in practice the formation of macromolecular assemblies will often be visible to the naked eye. For example, when a cloudy emulsion of polymer and lipid is prepared at a low counter ion concentration (such that the polymer is highly charged and most likely in the form of an extended chain), and the counter ion concentration is subsequently raised to a level where the hydrophilic/hydrophobic balance in the polymer chain is suitable for the formation of macromolecular assemblies (this pH level at which this effect occurs may be referred to as the ‘collapse pH’ at this particular concentration of counter ion) a noticeable solubilisation of lipid may be seen to occur which, depending on the quantities and exact nature of the individual components present, resulting in a marked partial or complete clearing of the mixture. The collapse pH refers to the pH level below which macromolecular assemblies may form in the case of polyanions. The reverse being true with polycations where a rise in pH is required to form the macromolecular assemblies.

At the lower end of the collapse pH range, the surface activity of 1:1 SMA is at its maximum. Such behaviour is consistent with the formation of an amphipathic structure in aqueous solution, such structure is able to associate with PL.

Without being limited by theory, it is believed that the pH at which the lipid interaction occurs is mainly dependent upon the attainment of a particular hydrophilic/hydrophobic balance within the polymer chains. Alternating polymers of styrene and maleic acid, as a result of their relatively high acid content, require a significant proportion of the acid functions to be neutralised before the correct hydrophilic/hydrophobic balance is obtained, only initiating an interaction with lipids at a pH in the region of 3 to 5 (collapse pH), and which interaction becomes unstable once the pH has been raised substantially above this level (for example above pH 5.5). Addition of 2% PEA to a 2.5% solution of 1:1 SMA will raise the collapse pH of the alternating copolymer to pH 8-9.

In light of this finding, it is possible to tailor the ratio of the 1:1 SMA to cationic counter ion concentration such that the polymer interacts with lipid over a specific but narrow pH range by variation of the counterion concentration the specific pH range over which this effect occurs can be modified, thereby enabling the selection of a SMA/counter ion concentration which is ideally suited for a chosen application, such applications can be over a wide range of different pH values.

Examples of Species Comprising a Counter Ion:

Embodiments of the invention include a species comprising a counter ion. Suitably the species comprising a counter ion is organic (i.e. consists of carbon, hydrogen, oxygen, nitrogen, sulphur, phosphorus and/or halogens, more suitably carbon, hydrogen, oxygen and/or nitrogen).

The counter ion may be cationic or anionic. A cationic counter ion has a net positive charge and an anionic counter ion has a net negative charge. In one embodiment the counter ion is provided by a basic or an acidic group. ‘Basic’ and ‘acidic’ refer to the charge carried by the group at pH 7. Generally a basic group has a positive charge at pH 7 and an acidic group has a negative charge at pH 7 dependent upon the pKₐ of the group in question.

In one embodiment, further ions are present in the composition. These further ions may be single or multivalent anions or cations (suitably cations) and are most suitably divalent, such as such as Ca²⁺ or Mg²⁺ ions. Alternatively, in one embodiment, substantially no further ions are present in the composition apart from the species comprising a counter ion (such as no further ions being present).

In one embodiment the species comprising a counter ion comprises a hydrophobic group. Suitably the hydrophobic group has a partially polarity and more particularly is charged but also possesses a substantially non polar character. Suitably the species comprising a counter ion comprises multiple hydrophobic groups, more suitably the species comprising a counter ion comprises one hydrophobic group.

In one embodiment the species comprising a counter ion is a hydrochloride, a sodium, a potassium, a sulphate, a acetate, a phosphate (or diphosphate), a chloride, or a maleate salt. More suitably, the species comprising a counter ion is a hydrochloride or a sodium salt. More suitably, the species comprising a counter ion is hydrochloride salt.

In one embodiment the species comprising a counter ion is selected from one or more salts of one or more of the following free bases:

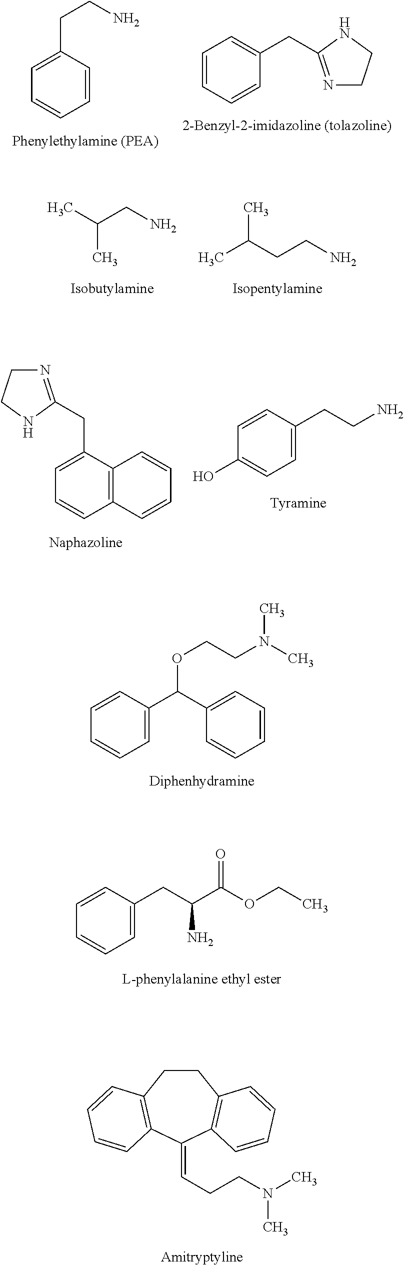

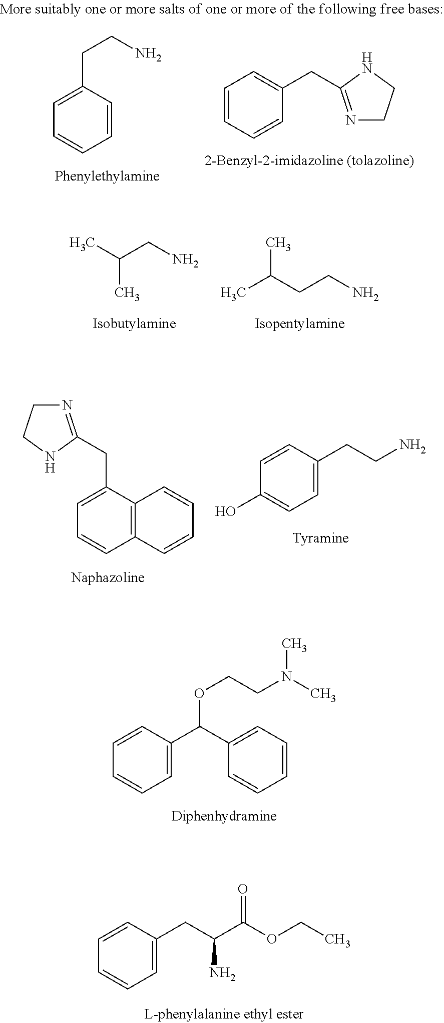

More suitably the salt is a hydrochloride, a sulphate, an acetate, a phosphate (or diphosphate), a chloride or a maleate salt. More suitably, a hydrochloride salt.

In one embodiment the species comprising a counter ion is not selected from one or more salts of one or more of the following free bases:

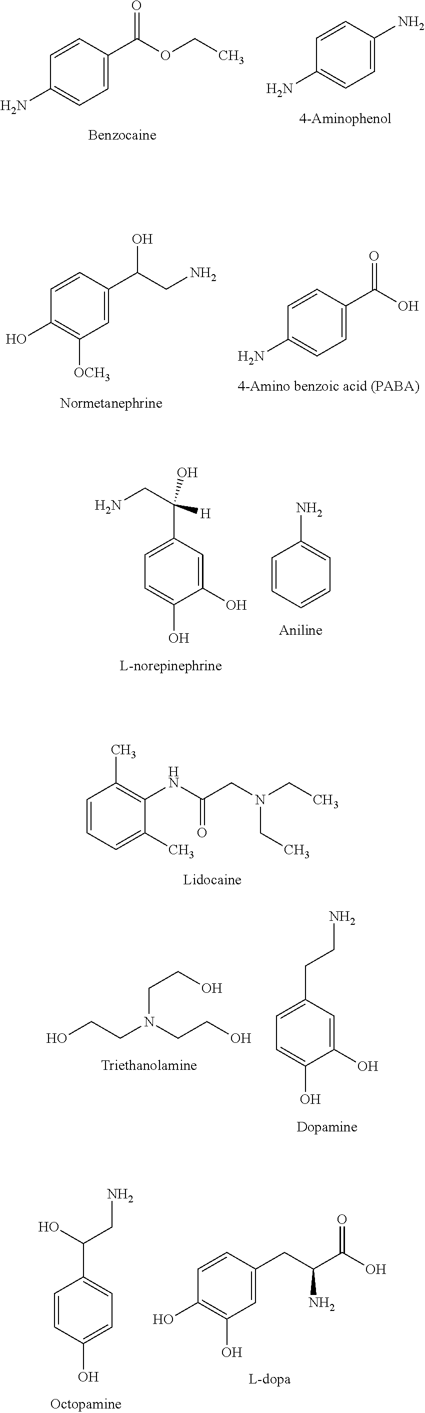

More suitably the salt is a hydrochloride, a sulphate, an acetate, a phosphate (or diphosphate), a chloride or a maleate salt. More suitably, a hydrochloride salt. More suitably, a hydrochloride salt.

When the counter ion is anionic then the associated cation salt is either sodium or potassium.

Examples of Polymers Used:

A particular example of suitable hydrophobically associating charged polymers are styrene/maleic acid copolymers (SMA). Styrene/maleic acid copolymers of use in the present invention will typically have an average molecular weight (M_{w}) of less than 500 kDa, especially less than 150 kDa, in particular less than 50 kDa and suitably less than 20 kDa (for example 1.5 to 15 kDa). M_{w}/Mₙ (Mₙ being the number average molecular weight) indicates the polydispersity, and will typically be less than 5, especially less than 4, in particular less than 3 and suitably less than 2. Polymers should be of sufficient length such that they may demonstrate the ability to form distinct hydrophilic and hydrophobic domains, but are suitably not so long as to introduce difficulties with viscosity as a result of interchain interactions.

The copolymer used in the present invention consists of a 1:1, preferably alternating, copolymer of styrene and maleic anhydride hydrolysed to maleic acid. A number of such polymers is available from Cray Valley Inc (USA) under the trade name SMA1000. Suitable grades are available as powder, flake or ultrafine powder preparations. Typical molecular weights as assessed by gel permeation chromatography (GPC). Alternatively such materials are available from Polyscope Polymers BV (Geleen, Netherlands) under the tradename Xiran.

Styrene/maleic anhydride copolymers must be hydrolysed for use in the present invention, and such hydrolysed polymers may optionally be used in the form of a salt. The polymers may be hydrolysed by a number of means, for example by reflux in aqueous solution, suitably in the presence of a strong base such as sodium hydroxide. Or more rapidly by microwave heating in aqueous solution. Partially hydrolysed styrene/maleic anhydride copolymers may also be of use in the present invention, however, in aqueous solution these are likely to hydrolyse further and for reasons of stability, fully hydrolysed polymer is typically used.

Certain salts of hydrolysed styrene/maleic anhydride copolymers are available commercially, for example sodium salts. Other salt forms are also available commercially, such as the ammonium or potassium salts. Although suitable for use in the present invention, ammonium salts are generally less desirable in pharmaceutical applications due to their associated odours.

A number of styrene/maleic anhydride copolymer half esters are commercially available. These esters may be hydrolysed for use in the present invention. Such half esters are available from Cray Valley Inc.

Commercial grades of the styrene/maleic anhydride copolymers, as supplied for industrial uses, may contain monomer, end-capping agent residuals and initiator residuals (e.g. maleic anhydride, styrene, cumene or p-cymene and acetophenone), such residuals are generally undesirable in compositions for use in pharmaceutical or biomedical products. Residual impurities may be removed or reduced in quantity by means known to those skilled in the art, such techniques include but are not limited to the selective solvation of the residual components into alcohols (for example methanol, ethanol or isopropanol) or into chlorinated solvents (for example chloroform or dichloromethane) or by repeated precipitation of the polymer in aqueous solution followed by crystallisation or alternatively by column chromatography.

Hydrolysed styrene/maleic anhydride copolymers, i.e. poly(styrene/maleic acid), and salts thereof (e.g. pharmaceutically acceptable salts, such as alkali metal salts, for example potassium or sodium) of use in the present invention will typically have a monomer ratio of styrene to maleic acid of 1:1.

Exemplary monomer ratios of use in the present invention include:1:1. In one embodiment of the invention the copolymer of styrene and maleic acid (or salt thereof) has an average molecular weight in the range 4,500 to 12,000 and a ratio of styrene to maleic acid of about 1:1.

Styrene/maleic acid copolymers with a monomer ratio of styrene to maleic acid of 1:1 may interact with lipids to form stable macromolecular assemblies at pH levels suitable for physiological use (e.g. within the pH ranges of 4-9) but may not necessarily demonstrate stable polymer and phospholipid macromolecular assemblies across this pH range although this can be easily obtained by modification of the concentration of the counter ion or species comprising a counter ion.

In one embodiment of the invention the polymer counter ion and lipid macromolecular assemblies are stable in aqueous solution at a pH between 4-9, especially between 7-8 (e.g. suitable for use in typical formulations for general application to the body and for association with proteins, especially membrane proteins, nucleotides, especially RNA and DNA).

In one embodiment of the invention the polymer counter ion and lipid macromolecular assemblies are stable in aqueous solution at a pH between 6.5-7.5 (e.g. suitable for use in typical formulations for general application to the body).

In one embodiment of the invention the polymer and lipid macromolecular assemblies are stable in aqueous solution at a pH between 7.1-7.8, especially between 7.3-7.6 (e.g. suitable for use in typical formulations for application to the eye).

Examples of Lipids and Cosurfactants Used:

Lipids of use in the present invention will typically be membrane forming lipids. Membrane forming lipids comprise a diverse range of structures including phospholipids (for example phosphatidyl choline, phosphatidyl ethanolamine, phosphatidyl glycerol, phosphatidyl inositol and phosphatidyl serine), ceramides and sphingomyelins, among others. Membrane forming lipids typically have a polar head group (which in a membrane aligns towards the aqueous phase) and one or more (e.g. two) hydrophobic tail groups (which in a membrane associate to form a hydrophobic core). The hydrophobic tail groups will typically be in the form of acyl esters, which may vary both in their length (for example from 8 to 26 carbon atoms) and their degree of unsaturation (for example one, two or three double bonds or more).

Lipids of use in the present invention may be of natural or synthetic origin, and may be a single pure component (e.g. 90% pure, especially 95% pure and suitably 99% pure on a weight basis), a single class of lipid components (for example a mixture of phosphatidyl cholines, or alternatively, a mixture of lipids with a conserved acyl chain type) or may be a mixture of many different lipid types.

In one embodiment of the invention the lipid comprises a phospholipid, more suitably essentially consists of a phospholipid, more suitably consists of a phospholipid.

In one embodiment of the invention the lipid is a single pure component.

Pure lipids are generally of synthetic or semi-synthetic origin. Examples of pure lipids of use in the present invention include phosphatidyl cholines (for example, DLPC, DMPC, DPPC and DSPC; in particular DLPC, DMPC and DPPC; such as DLPC and DPPC; especially DLPC) and phosphatidyl glycerols (for example DPPG), suitably phosphatidyl cholines. The use of pure lipids is desirable due to their defined composition, however, they are generally expensive.

In one embodiment of the invention the lipid is a mixture of components.

Mixtures of lipids of use in the present invention may be of natural origin, obtained by extraction and purification by means known to those skilled in the art. Lipid mixtures of natural origin are generally significantly cheaper than pure synthetic lipids. Naturally derived lipids include lipid extracts from egg or soy, which extracts will generally contain lipids with a mixture of acyl chain lengths, degrees of unsaturation and headgroup types. Exemplary lipid extracts of use in the present invention include:

S 75, S 100, S PC, SL 80 and SL-20-3; Phospholipon® 90 H, Phospholipon® 80 H, Phospholipon® 90 NG, available from Lipoid GmbH (Germany).

Lipid extracts of plant origin may typically be expected to demonstrate higher levels of unsaturation than those of animal origin. It should be noted that, due to variation in the source, the composition of lipid extracts may vary from batch to batch. Hydrogenated lipids are less prone to peroxidation due to the absence of unsaturation, typically have less coloration and have lower odour. Those from animal extracts, such as egg may be particularly suited for use in pharmaceutical formulations especially for use as injectable formulations.

In one embodiment of the invention the lipid is a lipid extract containing at least 50%, especially at least 75% and suitably at least 90% by weight of phospholipids of a single headgroup type (e.g. phosphatidyl cholines). In a second embodiment of the invention particular lipid extracts may be preferred due to their relatively cheap cost. In a third embodiment of the invention preferred lipid extracts are those which result in solutions of highest clarity. In a fourth embodiment of the invention the lipid is a lipid mixture having a conserved acyl chain length (e.g. at least 50%, especially at least 75% and suitably at least 90% by weight), for example 12 (e.g. lauryl), 14 (e.g. myristyl), 16 (e.g. palmityl) or 18 (e.g. stearyl) carbon atoms in length, in particular 12-16 (e.g. 14 or 16) carbon atoms. In another embodiment of the invention the lipid is a lipid mixture which is hydrogenated (i.e. the acyl chains are fully saturated).

Suitably, a lipid extract of use in the present invention will comprise at least 50% phospholipids by weight (for example, phosphatidyl cholines and phosphatidyl ethanolamines), especially at least 55% phospholipids by weight, in particular at least 60% phospholipids by weight (such as 75% or 90%).

One suitable lipid extract is derived from soy and comprises: at least 92% phosphatidyl cholines, a maximum of 3% lyso-phosphatidyl cholines and a maximum of 2% oils; of which 14-20% of the acyl chains are palmityl, 3-5% stearyl, 8-12% oleic, 62-66% linoleic and 6-8% linolenic. A second suitable lipid extract is derived from soy and comprises: at least 90% hydrogenated phosphatidyl cholines, a maximum of 4% hydrogenated lyso-phosphatidyl cholines and a maximum of 2% oils and triglycerides; of which at least 80% of the acyl chains are stearyl and at least 10% are palmityl.

Lipid mixtures may also be prepared by the combination of pure lipids, or by the combination of one lipid extract with either other lipid extracts or with pure lipids.

It may be desirable to utilise a lipid (either a pure lipid or a lipid mixture) which has a relatively low phase transition temperature, since this may facilitate preparation of compositions of the invention in the absence of heating.

For pharmaceutical applications typically the lipid (for example the pure lipid or the lipid mixture) is one which has been approved for use in pharmaceutical applications as appropriate.

Those skilled in the art will recognise that lipid mixtures of use in the invention may comprise non-membrane forming lipid components (e.g. cholesterol), or may in some circumstances be a mixture of only non-membrane forming lipids which in combination demonstrate membrane forming ability and a suitability for use in the invention. The latter being added as a means of modifying membrane fluidity to more accurately reflect those found in vivo and so maintain the native structure of membrane proteins contained within the polymer counter ion/PL macromolecular assemblies described herein.

In one embodiment of the invention the lipid is provided by the phospholipid membrane found in living cells, including both prokaryotic and eukaryotic cells which may be solubilised by the SMA polymer counter ion pairs described. In a further embodiment SMA polymer counter ion pairs can produce membrane pores in cell membranes to increase membrane permeability thereby acting as a conditioning agent for the membranes e.g. rending them permeable to certain drugs, nucleotides or other agents that need to be absorbed into the cell being treated with or exposed to the SMA polymer counter ion pairs described herein.

The presence of a small quantity of cosurfactant material may enhance the ability of the styrene/maleic acid copolymer counter ion pair to solubilise lipid (in particular lipid mixtures). This cosurfactant can take the form of a low molecular weight material, such as the naturally occurring lyso-phospatidyl choline (lyso-PC) which is available under the tradename S LPC from Lipoid GmbH. The cosurfactant may also be a combination of more than one surfactant. Suitable cosurfactant is added in an amount equivalent to between 0.1-5% of the weight of lipid in the composition, especially 0.5-2.5% and in particular 0.75-1.5% (for example about 1%). In one embodiment of the invention the cosurfactant is lyso-PC. It may be noted that certain lipid extracts may already contain lyso-PC, however, this does not preclude the addition of a cosurfactant.

Lyso-PC as a cosurfactant may be added either in its pure form (e.g. S LPC from Lipoid GmbH), or as one component of a lipid mixture (e.g. a high lyso-PC content lecithin, such as those having at least 10% lyso-PC content by weight, especially at least 15% lyso-PC by weight). An exemplary high lyso-PC content lecithin is SL20-3 from Lipoid GmbH.

Lipid mixtures (such as lipid extracts) which already contain a high lyso-PC content do not generally benefit significantly from the addition of further lyso-PC as a cosurfactant. As such, the need for a cosurfactant can be avoided simply by the selection of a lipid mixture which already contains a sufficient quantity of lyso-PC.

Examples of Polymer:Lipid and Polymer:Counter Ion Ratios:

The suitability of a particular pure lipid or lipid mixture for use in the present invention may be determined by those skilled in the art by routine experimentation based on the guidance provided herein.

Typically the ratio of polymer to lipid in the compositions of the present invention will be greater than 1:2 on a weight basis, especially greater than 1:1 (for example about 15:1 to 1.5: and particularly 2.5:1 or 1.5:1). Suitably the ratio of polymer to lipid in the compositions of the present invention will be greater than 1.25:1. Insufficient quantities of polymer may result in solutions with sub-optimal clarity. Excess quantities of polymer may result in an increased solution viscosity (which may or may not be a desirable feature depending upon the specific application). Suitably the ratio of polymer to lipid in the compositions of the present invention will be less than 100:1, such as less than 25:1, in particular less than 10:1 (e.g. less than 5:1).

The polymer:counter ion ratio on a weight basis is 20:1 to 1:1, typically 10:1 to 1:1 such as 10:1 to 5:1 or 5:1 to 1:1.

Example Compositions:

The compositions of the present invention may be in the form of an aqueous solution, especially a stable clear aqueous solution, suitably a stable clear and colourless aqueous solution. However, for ease of transportation and handling, once prepared, the compositions may be freeze-dried to form a dry powder which has the benefits of being lower in both volume and weight. In one embodiment of the present invention the composition is in the form of an aqueous solution. In a further embodiment of the present invention the composition is in freeze-dried form (for example as a powder, resin or flake, especially as a powder or flake, in particularly as a powder). Aqueous solutions include aqueous semi-solids such as gels and also fixed gels used for dry implants or depot release systems, or stent coatings.

In one embodiment of the invention there is provided an aqueous solution comprising 0.001-10% by weight of the compositions of the invention (the percentage being determined by the dry weight of composition of the invention relative to the total weight of composition and water). In a second embodiment of the invention there is provided an aqueous solution comprising 10-20% by weight of the compositions of the invention. In a third embodiment of the invention there is provided an aqueous solution comprising greater than 20% by weight of the compositions of the invention.

Compositions of the present invention may suitably be prepared by mixing a solution of a styrene/maleic acid copolymer, wherein the copolymer of styrene and maleic acid is alternating, adding a cationic counter ion salt and combining this with an aqueous emulsion containing lipid, and if necessary adjusting the cationic counter ion salt concentration or pH of the resulting mixture such that the polymer counter ion/PL macromolecular assemblies form.

Other compositions of the present invention may suitably be prepared by mixing a solution of a styrene/maleic acid copolymer having a ratio of styrene to maleic acid monomers of 1:1, with an aqueous solution of PEA hydrochloride salt and adding this mixture to an aqueous emulsion containing lipid, and if necessary adjusting the cationic counter ion PEA hydrochloride salt concentration and/or pH of the resulting mixture such that the polymer counter ion/PL macromolecular assemblies form.

The polymer solution may be prepared by dissolving the polymer in water, optionally with stirring and heating (for example to approximately 50° C.). The lipid emulsion may be prepared by mixing dried lipid with water under stirring and heating (suitably to a temperature above the phase transition temperature of the lipid component, for example approximately 50° C.), followed by homogenisation. Suitably the polymer cationic counter ion pair solution and lipid emulsion are mixed by the addition (e.g. the slow addition) of lipid emulsion to the polymer cationic counter ion pair solution, optionally together with heating (e.g. to around 50° C.).

The pH of solutions may be adjusted using acids or bases as appropriate. Compositions for use in the fields of pharmaceuticals and biomedical analysis will typically utilise acids and/or bases which are physiologically acceptable. Physiologically acceptable acids include hydrochloric acid. Physiologically acceptable bases include sodium or potassium hydroxide, suitably sodium hydroxide.

Cosurfactant, when present, will typically be mixed with lipid prior to the formation of the aqueous emulsion.

In a further aspect of the present invention there is provided a method for the production of a composition comprising lipid and a copolymer of styrene and maleic acid and a cationic counter ion, wherein the copolymer of styrene and maleic acid is alternating, wherein the polymer cationic counter ion and lipid are in the form of macromolecular assemblies, comprising the steps of:
(i) Preparing an aqueous solution of a copolymer of styrene and maleic acid, wherein the copolymer of styrene and maleic acid is alternating;
(ii) Preparing an aqueous solution of species comprising a cationic counter ion;
(iii) Mixing the species comprising a cationic counter ion solution to the aqueous solution of a copolymer of styrene and maleic acid;
(iv) Preparing an aqueous lipid emulsion;
(v) Mixing the aqueous lipid emulsion and aqueous solution of copolymer cationic counter ion;
(vi) Adjusting the concentration of species comprising a cationic counter ion or pH of the mixture, if necessary, such that polymer counter ion/PL macromolecular assemblies form.

In a further aspect of the present invention there is provided a method for the production of a composition comprising lipid and a copolymer of styrene and maleic acid and cationic counter ion, wherein the ratio of styrene to maleic acid monomer units is 1:1, wherein the polymer counter ion and lipid are in the form of macromolecular assemblies, comprising the steps of:
(i) Preparing an aqueous solution of a copolymer of styrene and maleic acid, wherein the copolymer of styrene and maleic acid is 1:1;
(ii) Preparing an aqueous solution of species comprising a cationic counter ion;
(iii) Mixing the species comprising a cationic counter ion solution to the aqueous solution of a copolymer of styrene and maleic acid;
(iv) Preparing an aqueous lipid emulsion;
(v) Mixing the aqueous lipid emulsion and aqueous solution of copolymer cationic counter ion;
(vi) Adjusting the concentration of species comprising a cationic counter ion or pH of the mixture, if necessary, such that polymer counter ion/PL macromolecular assemblies form.

If desirable, a further optional step of removing the water may be performed.

Compositions of the present invention in the form of an aqueous solution may be freeze-dried to produce compositions of the present invention in the form of a freeze-dried powder. Freeze-dried compositions may be readily reconstituted into aqueous solution by the addition of water with stirring and warming. The durability of compositions of the present invention to freeze-drying may be improved by the addition of protectants, for example sugars, such as trehalose (alpha, alpha-D-trehalose dihydrate, available from CMS Chemicals Ltd (UK)). Such freeze-dried compositions can additionally be combined with bulking agents such as maltodextrin, lactose or microcrystalline cellulose for preparation of pharmaceutical tablets or capsules.

Water may be removed by other means, such as rotary evaporation under reduced pressure and at an elevated temperature (e.g. 65-75° C.).

One use of compositions of the invention is as a solubilising agent or a stabilising agent for agents unstable in aqueous environments.

Solubilising agents may be of use as formulating aids, solubilising active agents which have poor aqueous solubility (for example aqueous solubility of less than 1% w/w, suitably less than 0.1% w/w or less than 0.01% w/w). Solubilising agents may also be of use as carriers for active agents which preferentially partition into the solubilising agent (for example, active agents which partition into octanol as opposed to water, i.e. are predominantly hydrophobic in nature). The active agent may be a medicament for the treatment or prevention of a medical disorder.

Active agents having poor aqueous solubility and/or stability include the oil-soluble vitamins (including vitamins A). The vitamin A family includes retinol, retinal, retinol, esters such as retinol acetate or retinol propionate, and related retinoids. Other oil-soluble actives based upon a steroidal structure include those used to treat inflammatory conditions (such as hydrocortisone, clobetasone butyrate, hydrocortisone butyrate, clobetasol propionate, fluticasone propionate and dexamethasone, in particular hydrocortisone, clobetasone butyrate, hydrocortisone butyrate, clobetasol propionate and dexamethasone) and hormones (such as testosterone, anabolic steroids, oestrogen and oestrogens). Additional steroidal compounds include dexamethasone acetate anhydride, hydrocortisone acetate and cortisone acetate.

Other water insoluble or poorly soluble actives include cannabinoids such as; CBD (cannabidiol), THC (tetrahydrocannabinol), CBDA (cannabidiolic acid), CBG (cannabigerolic acid), CBN (cannabinol), CBC (cannabichromene) and THCV (tetrahydrocannabivarin).

Still further water insoluble or poorly soluble or membrane-active agents include antimicrobials: antibacterials, such as erythromycin, neomycin (e.g. as the sulphate), poly-c-lysine, nisin A, colistimethate sodium, polymixin B sulphate, colistin, daptomycin and linezolid; antifungals, such as ciclopirox olamine, piroctone olamine (each of which are an example of pyridone antifungals), clotrimazole, econazole, ketaconazole and nystatin (in particular piroctone olamine, clotrimazole, ketaconazole and nystatin).

The quantity of active agent which may be combined with and solubilised in the compositions of the present invention will typically be in the range of 0.001-50% of the weight of polymer counter ion pair and lipid, especially in the range of 0.001-25% (e.g. 5-20%).

Active agents may be conveniently incorporated into the compositions of the present invention by the addition of the active agent to the lipid (and where appropriate to the lipid and cosurfactant) prior to the preparation of the aqueous lipid emulsion, and before the emulsion and polymer counter ion pair solution are mixed.

There is provided an aqueous formulation comprising a composition of the invention, and which further comprises an active agent.

In an analogous manner to compositions of the invention, aqueous formulations of the present invention (which comprise an active agent) may generally be freeze-dried and reconstituted as necessary. As such, also provided is a formulation comprising a composition of the invention, and which further comprises an active agent, which is in freeze-dried form (for example as a powder, resin or flake, in particular powder or flake).

In general a formulation of the present invention will be incorporated into a pharmaceutical preparation which is tailored to suit the particular purpose, manner of use and mode of administration. Formulations may be mixed with one or more pharmaceutically acceptable carriers or excipients (anti-oxidants, preservatives, viscosity modifiers, colourants, flavourants, buffers, acidity regulators, chelating agents, or other excipients), and optionally with other therapeutic ingredients if desired. Such preparations may be prepared by any of the methods known in the art, and may for example be designed for inhalation, topical or parenteral (including intravenous, intra-articular, intra-muscular, intra-dermal and subcutaneous) administration.

Preparations for systemic delivery are suitably made using low molecular weight copolymer, although this polymeric material is non-degradable, the butyl half ester has previously been used in medicine (known as SMANCS) and is likely to be readily excreted through the kidneys or through the liver/bile. The species comprising the counter ions described in this application are used as nutritional supplements or drugs, while some of the phospholipids described in this application are used for parenteral nutrition and are likely to be readily broken down in the body without causing serious problems. Preparations for parenteral delivery will suitably be sterile.

There is also provided a pharmaceutical preparation comprising a composition of the invention and an active agent, and which further comprises a pharmaceutically acceptable carrier or excipient.

There is also provided a sterile composition prepared by filtration.

Accordingly, there is also provided a composition of the invention for use in therapy.

There is also provided a pharmaceutical composition for cellular and intracellular delivery and for delivery across membrane barriers such as the gut:blood and blood:brain barriers.

Use for Membrane Protein Extraction and Characterisation:

Other potential uses of compositions of the present invention include as a means of solubilising membrane peptides or proteins for the investigation of their structure. A need has been identified for solubilising agents that can be used for solubilising membrane peptides and proteins (including integral, membrane tethered or membrane associated proteins, for example drug receptor proteins or GPCRs), within phospholipid membranes in such a way as to retain their native conformation and thereby to enable their structure to be investigated by spectroscopic means (e.g. by NMR spectroscopy, Mass spectroscopy).

In addition to structural investigations, it may also be desirable to investigate the interactions of membrane proteins and peptides with other species. Such other species may also be membrane peptides and proteins, nucleotides or oligonucleotides such as RNA and DNA. In the case of membrane receptors such other species include ligands and ligand fragments (e.g. drug agonists and antagonists). In the case of enzymes, such other species may be ligands and ligand fragments (e.g. substrate(s) and inhibitors).

Other membrane bound or membrane associated molecules which may be the subject of investigations include glycolipids and immunoglobulins.

In addition to NMR and MS, there are many other suitable spectroscopic techniques which are well known to those skilled in the art for the purposes of investigating peptides and proteins (including x-ray crystallography, infra-red spectroscopy, cryo-electron microscopy, circular dichroism and UV spectroscopy).

Numerous techniques exist for the transfer of membrane proteins from a detergent solubilised state to a lipid bilayer state. The disadvantage of such techniques is that they result in denaturation of the protein structure and unfolding leading to a loss of membrane protein function.

Compositions of the present invention may offer an advantage over the use of detergents and bicelles³¹ for the purpose of reconstituting membrane peptides and proteins in a fully functional state.
31. Sanders, C R & Landis, G C. Reconstitution of membrane proteins into lipid-rich bilayered mixed micelles for NMR studies. Biochemistry. 34(12), 4030-4040 (1995).

Accordingly, there is provided the use of a composition of the invention for the solubilisation of a membrane peptide or protein. Also provided are compositions of the invention (e.g. in dry or aqueous form) which further comprise a membrane peptide or protein.

Further, there is provided a method for the screening of candidate agents for interaction with a membrane protein or peptide comprising the steps of:
(i) solubilising a membrane protein or peptide in a composition of the invention;
(ii) testing a candidate agent to determine whether it interacts with the solubilised membrane protein or peptide.

Candidate agents may be putative ligands or ligand fragments (e.g. drug agonists, antagonists, inhibitors and such).

It may also be envisaged that the compositions of the present invention may be used to provide a platform to maintain specific proteins in cell-free media for use as processing aids, as catalytic or enzyme systems in the production of biological actives e.g. in fermentation/reaction vessels for industrial production or for incorporation into hybrid solar devices for the photochemical production of sustainable electrical energy or carbon capture, especially carbon dioxide, and recycling.

It may also be envisaged that the compositions of the present invention may be used to solubilise peptides or proteins which are immunogenic in nature (e.g. antigens). Alternatively, it may be noted that WO95/11700³² discloses an oil-in-water submicron emulsion (SME) for use as a vaccine adjuvant for enhancing immunogenicity and improving the immune response of antigens in vaccines. Compositions of the present invention may also be of use as particulate vaccine adjuvants.
32. Lowell, G H, Amselem, S & Friedman, D, Submicron Emulsions as Vaccine Adjuvants. WO95/11700, 4 May 1995 to Pharmos Corp, New York, N.Y., USA.

Furthermore, there is a need for treatment of medical conditions affecting mucosal surfaces, e.g. for ophthalmic use in the treatment of the condition known as “dry eye” syndrome, and for lubricating biological membranes (e.g. synovial). The tear film has a coating of phospholipids, which are necessary for the formation of a stable tear film. Diseases where the tear film is deficient may potentially be treated by the addition of an aqueous phospholipid solution, such as an aqueous solution of the compositions of the present invention. Compositions of the present invention are advantageous in this regard, since they are clear and colourless, and therefore suitable for use in eye drops, unlike conventional aqueous preparations of phospholipids which may be opaque.

There is also a need for lubricating phospholipids to treat the surfaces of articulated joints in connection with arthritic conditions or to lubricate surfaces of medical devices and prostheses, e.g. artificial joints and contact lenses, that are fitted into or onto the body, or to prevent focal adhesions between tissues such as those that may occur during surgical procedures. Compositions of the present invention may be of use in this regard (e.g. by intra-articular injection).

The compositions of the invention may also have the ability to deliver active agents locally to the lung or, via the highly permeable membranes lining the deep lung, into the systemic circulation. The similarity between the bilayer phospholipid compositions of the invention and the multi-lamellar surfactant fluid lining the internal alveolar and bronchial surfaces of the lung make compositions of the invention particularly suited to deliver active agents to the lung, especially the deep lung, or to act as a means of delivering phospholipid (e.g. DPPC) to the lung for the treatment of neonatal or adult respiratory distress syndrome, a condition characterised by a insufficient levels of native lung surfactant or phospholipid. Delivery to the lung may be by aerosol or by nebulisation.

In a further embodiment of the invention, there is provided a composition consisting essentially of, or more suitably consisting of, a lipid, a hydrophobically associating charged polymer and a species comprising a counter ion, wherein the counter ion is oppositely charged to the polymer and wherein the species comprising a counter ion further comprises a hydrophobic group.

Clauses setting out further embodiments of the invention are as follows:
1. A composition comprising a lipid, a hydrophobically associating charged polymer and a species comprising a counter ion, wherein the counter ion is oppositely charged to the polymer.
2. The composition of clause 1 wherein the species comprising a counter ion is organic.
3. The composition of either clause 1 or 2 wherein the hydrophobically associating charged polymer is anionic and the counter ion is cationic.
4. The composition of clause 3 wherein the counter ion is provided by a basic group.
5. The composition of clause 4 wherein the basic group is a functional group comprising nitrogen and/or sulphur.
6. The composition of clause 5 wherein the basic group is a functional group comprising nitrogen.
7. The composition of clause 6 wherein the functional group is selected from one or more of an, an amine, an imine, an imide, an azide, an azo compound, or salts thereof.
8. The composition of clause 7 wherein the functional group is an amine or salt thereof.
9. The composition of either clause 7 or 8 wherein the salt is a hydrochloride salt.
10. The composition of any one of clauses 1 to 9 wherein the hydrophobically associating charged polymer comprises carboxylic acid groups.
11. The composition of any one of clauses 1 to 9 wherein the hydrophobically associating charged polymer is selected from a copolymer of styrene and maleic acid, a polymer of methacrylic acid, a polymer of itaconic acid, a polymer of maleic acid or a copolymer of diisobutylene and maleic acid.
12. The composition of clause 11 wherein the hydrophobically associating charged polymer is selected from a copolymer of styrene and maleic acid, a polymer of methacrylic acid, a polymer of itaconic acid or a polymer of maleic acid.
13. The composition of clause 12 wherein the hydrophobically associating charged polymer is a copolymer of styrene and maleic acid.
14. The composition of clause 13 wherein the copolymer of styrene and maleic acid is alternating.
15. The composition of either clause 13 or 14 wherein the ratio of styrene to maleic acid monomer units is 1:1 or greater.
16. The composition of clause 15 wherein the ratio of styrene to maleic acid monomer units is 1:1 to 5:1.
17. The composition of clause 16 wherein the ratio of styrene to maleic acid monomer units is 1:1 to 3:1.
18. The composition of clause 17 wherein the ratio of styrene to maleic acid monomer units is 1:1.
19. The composition of either clause 1 or 2 wherein the hydrophobically associating charged polymer is cationic and the counter ion is anionic.
20. The composition of clause 19 wherein the counter ion is provided by an acidic group.
21. The composition of clause 20 wherein the acidic group is selected from one or more of a nitrate, a nitrile, a nitrite, a nitro compound, a nitroso compound, a carboxylate, a phosphate, a sulphate, a sulphite, a cyanate, or salts thereof.
22. The composition of clause 21 wherein the salt is a sodium or potassium salt.
23. The composition of clause 22 wherein the salt is a sodium salt.
24. The composition of clause 19 wherein the hydrophobically associating charged polymer is a copolymer of styrene and maleimide.
25. The composition of any one of clauses 1 to 24 wherein the species comprising a counter ion comprises a hydrophobic group.
26. The composition of clause 25 wherein the hydrophobic group comprises or consists of an aryl group.
27. The composition of clause 26 wherein the aryl group is a phenyl group.
28. The composition of clause 26 wherein the aryl group is a 6-membered aromatic ring containing at least one heteroatom, such as one nitrogen atom (pyridinyl), two nitrogen atoms (pyridazinyl, pyrimidinyl or pyrazinyl) and three nitrogen atoms (triazinyl).
29. The composition of clause 28 wherein the aryl group is a 6-membered aromatic ring containing one nitrogen atom (pyridinyl).
30. The composition of any one of clauses 25 to 29 wherein the hydrophobic group comprises or consists of an aliphatic group (such as an alkyl or alkenyl group).
31. The composition of clause 30 wherein the alkyl group consists of branched or unbranched C₁-C₁₀ alkyl.
32. The composition of clause 31 wherein the alkyl group consists of branched or unbranched C₁-C₅ alkyl.
33. The composition of clause 32 wherein the alkyl group consists of C₁-C₃ alkyl.
34. The composition of clause 33 wherein the alkyl group is ethyl or C₂H₄.
35. The composition of clause 30 wherein the alkenyl group consists of branched or unbranched alkenyl.
36. The composition of clause 35 wherein the alkenyl group consists of branched or unbranched C₁₋₅ alkenyl.
37. The composition of any one of clauses 25 to 36 wherein the species comprising a counter ion comprises a linker connecting the hydrophobic group to the basic or acidic group.
38. The composition of clause 37 wherein the linker comprises or consists of an alkyl or alkenyl group.
39. The composition of clause 38 wherein the alkyl group consists of branched or unbranched C₁-C₁₀ alkyl.
40. The composition of clause 39 wherein the alkyl group consists of branched or unbranched C₁-C₅ alkyl.
41. The composition of clause 40 wherein the alkyl group consists of C₁-C₃ alkyl.
42. The composition of clause 41 wherein the alkyl group is ethyl or C₂H₄.
43. The composition of clause 42 wherein the species comprising a counter ion is phenethylamine (PEA).
44. The composition of clause 38 wherein the alkyl group consists of branched or unbranched alkenyl.
45. The composition of clause 44 wherein the alkyl group consists of branched or unbranched C₁₋₅ alkenyl.
46. The composition of any one of clauses 1 to 45 wherein the species comprising a counter ion is present at a mass of between about 50-250 Da.
47. The composition of clause 46 wherein the species comprising a counter ion is present at a mass of between about 100-250 Da.
48. The composition of clause 47 wherein the species comprising a counter ion is present at a mass of between about 120-170 Da.
49. The composition of any one of clauses 1 to 48 wherein the species comprising a counter ion is present at a concentration of between about 0.1-2.0% w/w.
50. The composition of clause 49 wherein the species comprising a counter ion is present at a concentration of between about 0.2-0.5% w/w.
51. The composition of clause 50 wherein the species comprising a counter ion is present at a concentration of between about 0.25-0.4% w/w.
52. The composition of any one of clauses 3 to 18 or 24 to 51 wherein the species comprising a counter ion, in its acid form, has a pKa of about 7-12.
53. The composition of clause 52 wherein the species comprising a counter ion, in its acid form, has a pKa of about 8-11.
54. The composition of clause 53 wherein the species comprising a counter ion, in its acid form, has a pKa of about 9.5-10.5.
55. The composition of clause 54 wherein the species comprising a counter ion, in its acid form, has a pKa of about 9.8.
56. The composition of any one of clauses 19 to 51 wherein the species comprising a counter ion, in its base form, has a pKb of about 11-8.
57. The composition of clause 56 wherein the species comprising a counter ion, in its base form, has a pKb of about 10.5-9.
58. The composition of clause 56 wherein the species comprising a counter ion, in its base form, has a pKb of about 10-9.
59. The composition of clause 56 wherein the species comprising a counter ion, in its base form, has a pKb of about 9.5.
60. The composition of any one of clauses 1 to 59 wherein the species comprising a counter ion is not a metal ion.
61. The composition of any one of clauses 1 to 60 wherein the species comprising a counter ion is selected from one or more of the hydrochloride salts of the free bases shown:

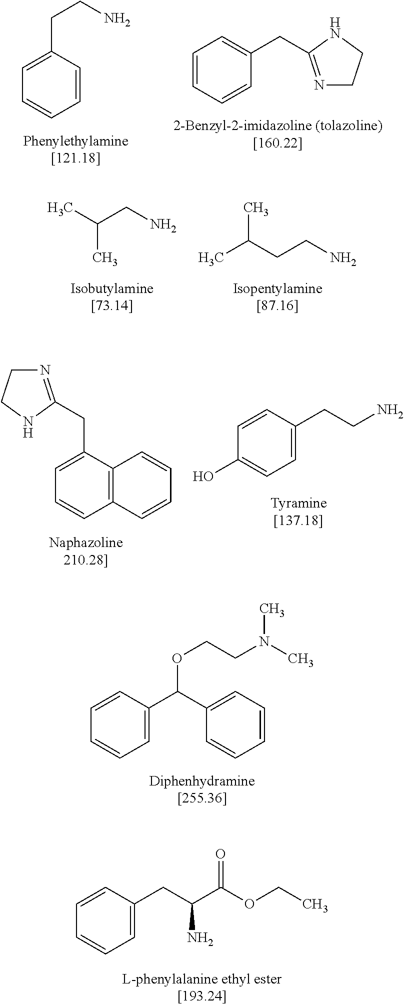

62. The composition of any one of clauses 1 to 61 wherein the counter ion is not one of the hydrochloride salts of the free bases shown:

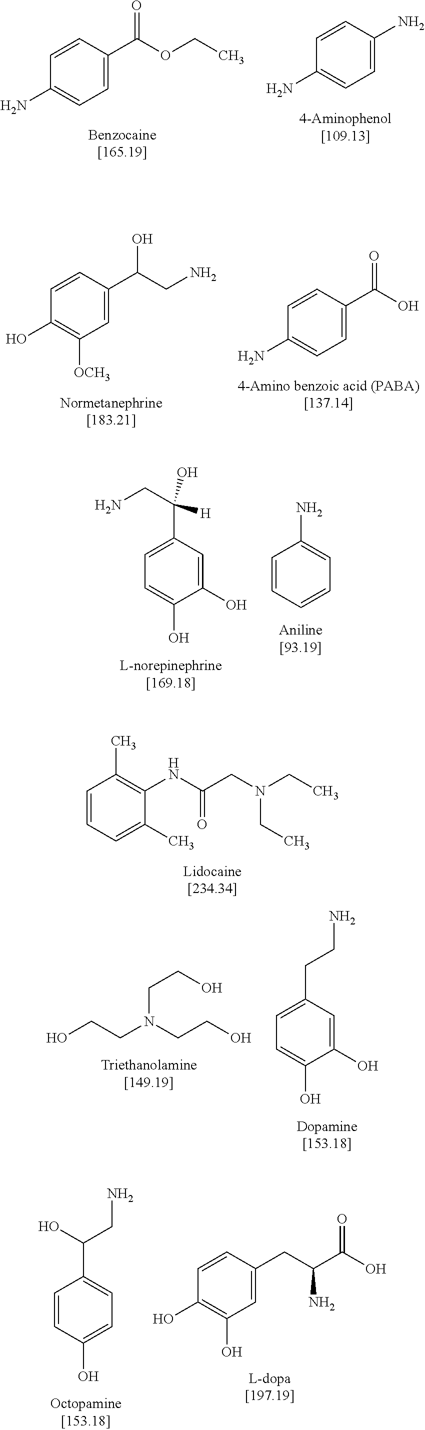

[Mol Wt of free base]
63. The composition of any one of clauses 1 to 62 wherein the polymer and lipid are in the form of macromolecular assemblies.
64. A composition according to any one of clauses 1 to 63, wherein the polymer has an average molecular weight of less than 500,000 daltons.
65. A composition according to clause 64, wherein the polymer has an average molecular weight of less than 150,000 daltons.
66. A composition according to clause 65, wherein the polymer has an average molecular weight of less than 50,000 daltons.
67. A composition according to clause 66, wherein the polymer has an average molecular weight of less than 20,000 daltons.
68. A composition according to any one of clauses 13 to 67 wherein the polymer is a copolymer of styrene and maleic acid and has an average molecular weight in the range 4,500 to 12,000 and a ratio of styrene to maleic acid of about 1:1, 2:1, 3:1 or 4:1.
69. A composition according to any one of clauses 1 to 68, wherein the composition is stable in aqueous solution at a pH between 7-8.5.
71. The composition of any one of clauses 1 to 69 wherein the lipid is a phospholipid.
72. A composition according to any one of clauses 1 to 71, wherein the lipid is a single pure component.
73. A composition according to clause 72, wherein the single pure component is a phosphatidyl choline.
74. A composition according to clause 73, wherein the phosphatidyl choline is DLPC,
DMPC, DPPC or DSPC.
75. A composition according to clause 74, wherein the phosphatidyl choline is DLPC.
76. A composition according to clause 72, wherein the single pure component is a phosphatidyl glycerol.
77. A composition according to clause 76, wherein the phosphatidyl glycerol is DPPG.
78. A composition according to any one of clauses 1 to 71, wherein the lipid is a mixture of components.
79. A composition according to clause 78, wherein the lipid is a lipid mixture having a conserved acyl chain length.
80. A composition according to clause 79, wherein the conserved acyl chain length is 12, 14, 16, or 18 carbon atoms in length.
81. A composition according to clause 80, wherein the conserved acyl chain length is 12-16 carbon atoms in length.
82. A composition according to clause 78, wherein the lipid is a lipid mixture of at least 50% phospholipids having a single headgroup type by weight.
83. A composition according to clause 82, wherein the lipid is a lipid mixture of at least 75% phospholipids having a single headgroup type by weight.
84. A composition according to clause 83, wherein the lipid is a lipid mixture of at least 90% phospholipids having a single headgroup type by weight.
85. A composition according to any one of clauses 82 to 84, wherein the single headgroup type is a phosphatidyl choline.
86. A composition according to any one of clauses 1 to 85, wherein the lipid is a lipid extract of natural origin.
87. A composition according to clause 86, wherein the lipid extract is derived from egg.
88. A composition according to clause 86, wherein the lipid extract is derived from soy.
89. A composition according to any one of clauses 1 to 88, wherein the ratio of polymer to lipid is greater than 1:2 on a weight basis.
90. A composition according to clause 89, wherein the ratio of polymer to lipid is greater than 1:1 on a weight basis.
91. A composition according to clause 90, wherein the ratio of polymer to lipid is about 1.5:1 on a weight basis.
92. A composition according to clause 91, wherein the ratio of polymer to lipid is about 2.5:1 on a weight basis.
93. A composition according to any one of clauses 1 to 92, which further comprises a cosurfactant.
94. A composition according to clause 93, which cosurfactant is added in an amount equivalent to 0.1 to 5% of the weight of lipid in the composition.
95. A composition according to clause 94, which cosurfactant is added in an amount equivalent to 0.5 to 2.5% of the weight of lipid in the composition.
96. A composition according to clause 95, which cosurfactant is added in an amount equivalent to 0.75 to 1.5% of the weight of lipid in the composition.
97. A composition according to clause 96, which cosurfactant is added in an amount equivalent to about 1% of the weight of lipid in the composition.
98. A composition according to any one of clauses 93 to 97, wherein the cosurfactant is lyso-phosphatidyl choline.
99. A composition according to any one of clauses 1 to 98, wherein the macromolecular assemblies are less than 100 nm in diameter.
100. A composition according to clause 99, wherein the macromolecular assemblies are less than 50 nm in diameter.
101. A composition according to clause 100, wherein the macromolecular assemblies are less than 25 nm in diameter.
102. A composition according to any one of clauses 1 to 101, which is in freeze-dried form.
103. An aqueous solution comprising a composition according to any one of clauses 1 to 101.
104. An aqueous solution according to clause 103, comprising 0.001-10% by weight of a composition according to any one of clauses 1 to 101.
105. An aqueous solution according to clause 104, comprising 10-20% by weight of a composition according to any one of clauses 1 to 101.
106. An aqueous solution according to clause 105, comprising greater than 20% by weight of a composition according to any one of clauses 1 to 101.
107. An aqueous solution according to any one of clauses 103 to 106, which is clear and stable and has a pH between 2-9.
108. An aqueous solution according to clause 107, which has a pH of 2-9.
109. An aqueous solution according to clause 108, which has a pH of 7-8.5.
110. An aqueous solution according to any one of clauses 103 to 109, which has a pH of 7.1-7.8.
111. An aqueous solution according to clause 110, which has a pH of 7.3-7.6.
112. An aqueous solution according to any one of clauses 103 to 111, which is clear.
113. An aqueous solution according to any one of clauses 103 to 112, which is colourless.
114. An aqueous solution according to any one of clauses 103 to 113, which is stable.
115. A formulation comprising a composition according to any one of clauses 1 to 114 or an aqueous solution according to any one of clauses 103 to 114, which further comprises an active agent.
116. A formulation according to clause 115, wherein the active agent is an oil soluble vitamin or oil soluble vitamin derivative.
117. A formulation according to clause 116, wherein the oil soluble vitamin or oil soluble vitamin derivative is a retinoid, vitamin A, retinol, retinaldehyde or retinoic acid.
118. A formulation according to clause 115, wherein the active agent has a triterpenoid or steroidal nucleus.
119. A formulation according to clause 115, wherein the active agent is a peptide.
120. A formulation comprising a composition according to any one of clauses 1 to 119 or an aqueous solution according to any one of clauses 103 to 114, which further comprises a membrane peptide or protein.
121. A composition, aqueous solution or formulation according to any one of clauses 1 to 120 for use in therapy.
122. A pharmaceutical preparation comprising composition, aqueous solution or formulation according to any one of clauses 1 to 120, which further comprises a pharmaceutically acceptable carrier or excipient.
123. Use of a composition, aqueous solution or formulation according to any one of clauses 1 to 120 as a solubilising agent.
124. Use according to clause 123 for the solubilisation of an active agent.
125. Use according to clause 124, wherein the active agent is an oil soluble vitamin or oil soluble vitamin derivative.
126. Use according to clause 125, wherein the active agent has a triterpenoid or steroidal nucleus.
127. Use according to clause 124, wherein the active agent is a peptide.
128. Use according to clause 124 to solubilise a membrane peptide or protein or lipid soluble peptide or protein.
129. Use of a composition, aqueous solution or formulation according to any one of clauses 1 to 120, in the manufacture of a pharmaceutical preparation.
130. A method for the production of a composition according to any one of clauses 1 to 102 comprising the steps of:
   (i) Preparing an aqueous solution of a hydrophobically associating charged polymer and a counter ion;
   (ii) Preparing an aqueous lipid emulsion;
   (iii) Mixing the lipid emulsion and aqueous solution of polymer and counter ion;
   (iv) Adjusting the pH of the mixture, if necessary, such that polymer/lipid macromolecular assemblies form;
   (v) Optionally removing the water.
131. A method for the production of a formulation according to any one of clauses 115 to 120 comprising the steps of:
   (i) Preparing an aqueous solution of a hydrophobically associating charged polymer and a counter ion;
   (ii) Preparing an aqueous emulsion of lipid and active agent;
   (iii) Mixing the aqueous emulsion and aqueous solution of polymer and counter ion;
   (iv) Adjusting the pH of the mixture, if necessary, such that polymer/lipid macromolecular assemblies form;
   (v) Optionally removing the water.
132. A method of solubilising a lipid in aqueous solution comprising the formation of macromolecular assemblies of the lipid and a hydrophobically associating charged polymer and a counter ion.
133. A method of solubilising an active agent having poor aqueous solubility in aqueous solution comprising the formation of macromolecular assemblies of the lipid, active agent and a hydrophobically associating charged polymer and a counter ion.
134. A method for the screening of candidate agents for interaction with a membrane protein or peptide comprising the steps of:
   (i) solubilising a membrane protein or peptide in a composition comprising a lipid, a hydrophobically associating charged polymer and a counter ion, wherein the polymer and lipid are in the form of macromolecular assemblies;
   (ii) testing a candidate agent to determine whether it interacts with the solubilised membrane protein or peptide.
135. The composition, aqueous solution, formulation, pharmaceutical preparation, use or method according to any one of clauses 1-134 wherein the species comprising a counter ion consists of (a) a basic group or acid group, (b) a hydrophobic group and (c) a linker connecting the hydrophobic group to the basic or acidic group.

The following Examples are non-limiting and are provided to illustrate the preparation and use of compositions according to the present invention such that a person skilled in the art may more readily appreciate the nature of the invention and put the invention into practical effect.

### COMPARATIVE EXAMPLES

### Comparative Example 1: The Ability of Polymer (SMA) Counter Ion Pair to Solubilise Lipid

Lipids

DLPC (1,2-Dilauroyl-sn-glycero-3-phosphorylcholine), CAS Number 18194-25-7, was obtained at 99% purity, synthetic, from Sigma-Aldrich Chemical Company, Merck KGaA, Darmstadt, Germany.

Polymers

SMA1000P was obtained from Cray Valley Inc. (USA) and contains a 1:1 ratio of styrene to maleic anhydride monomer units. The polymer is supplied in powder form, in an unhydrolysed state and was hydrolysed to the maleic acid form prior to use.

Results

Table 1 below summarises the results of the experiment. Particle Size Studies (see Table 1 for experimental details) show the following particle sizes established using a Particlemetrix Laser Scattering Microscope (Germany):
1) SMA-PL macromolecular assemblies (Test), SMA 1:1 plus 8 mg PEA (i.e. PEA HCl at 0.4% w/w of solution or 16% w/w of the polymer (2.5% w/w)) at pH 8=54.2 nm diameter.
2) SMA-PL macromolecular assemblies (Control), SMA 1:1 at pH 4−5=93.5 nm diameter.

### Comparative Example 2: The Ability of Different Counter Ions to Form Polymer Counter Ion/PL Macromolecular Assemblies

Percentage values specified in this experiment refer to the weight of the component in question as a proportion of the total weight of the composition.

Mixtures were visually examined to determine whether the polymer counter ion component had solubilised the lipid component in the aqueous medium. The clarity of a mixture was categorised as being clear if there was no significant visible opacity to the naked eye.

Results

Table 1 below summarises the results of the experiments.

### Comparative Example 3: Composition of the Invention

Method

A stock emulsion of membrane forming lipid was prepared at double the desired final concentration. Lipid was added to the appropriate volume of water, followed by stirring and heating to approximately 50° C. until a uniform emulsion was formed. A stock solution of polymer was prepared at double the desired final concentration. Polymers which were supplied as styrene/maleic anhydride were hydrolysed by refluxing in water for two hours in the presence of excess sodium hydroxide, before being left at 4° C. for 48 hours to ensure that the reaction was complete. Stock solutions were prepared by mixing of the hydrolysed polymer with the appropriate volume of water and adjusted to pH 8 with the addition of 1M sodium hydroxide solution.

The species comprising counter ion salts were prepared as 10% w/w aqueous solutions and adjusted to chosen pH with the addition of 1M sodium hydroxide solution and the resulting solution added dropwise to the solution of polymer previously prepared.

Polymer counter ion pair/PL mixtures were then prepared by the dropwise addition of the lipid emulsion to twice the concentration of polymer counter ion solution while stirring and heating to approximately 50° C.

The pH of the resulting mixtures were maintained at approximately pH 8, in the case of those mixtures which had not produced clear and colourless solutions at this point, pH was lowered until the solution cleared by the addition of 1M hydrochloric acid added dropwise.

Percentage values specified in this experiment refer to the weight of the component in question as a proportion of the total weight of the composition.

Mixtures were visually examined to determine whether the polymer counter ion pair component had solubilised the lipid component in the aqueous medium. The clarity of a mixture was categorised as being clear if there was no significant visible opacity to the naked eye, whereas a mixture was categorised as cloudy if there was significant visible disruption to the passage of light.

Polymers

SMA1000P was as described in Comparative Example 1.

Poly(Itaconic acid) zinc salt was obtained from Croda UK, Goole, UK.

Poly(methacrylic acid) as the sodium salt of 4-6k mol wt was obtained from supplied by Sigma Aldrich, Poole, UK.

Poly(maleic acid) of 0.8-1.2k mol wt was obtained from Polysciences Inc. Warrington, USA.

Species Comprising Cationic Counter Ion

Supplied as salts or free bases converted to salts by addition of 1M hydrochloric acid. 2-Phenylethylamine hydrochloride (PEA), CAS Number 156-28-5, was obtained at 99% purity from Blackburn Distributions (UK).

Isopentyl amine, tyramine, diphenhydramine hydrochloride, phenylalanine ethyl ester hydrochloride, 2-benzyl-2-imidazoline and naphazoline hydrochloride were all obtained from Sigma-Aldrich Chemical Company, Merck KGaA, Poole, England.

Species Comprising Anionic Counter Ion Supplied as free acid converted to salts by addition of 1M sodium hydroxide.

Benzoic acid was obtained by S. Black Limited, Hertford, England.

Lipids

DLPC (1,2-Dilauroyl-sn-glycero-3-phosphorylcholine), is described in Comparative Example 1.

Results

Table 1 below summarises the results of experiments.

A full description of many of the components utilised in this supplemental experiment is available elsewhere in the Examples.

TABLE 1
Results of Experiments

| | | | | | | | Species | | | Stability* |
|---|---|---|---|---|---|---|---|---|---|---|
| | | | | | Final | | comprising | Amine | pH for | After 2-4 |
| | Polymer | Polymer | DLPC | Ratio | Vol | Start | counter ion | HCl | clear | mths + at |
| Polymer | type/S:M | (mg) | (mg) | Polymer:Lipid | (ml) | pH | (Amine HCl) | [% w/w] | sample | 4° C. |
| SMA | 2:1 | 50 | 25 | 2:1 | 2 | | PEA | 40 mg | 9 clear | |
| | | | | | | | | [2] | | |
| SMA | 1:1 | 50 | 25 | 2:1 | 2 | | PEA | 40 mg | 8.5 clear | PS |
| | | | | | | | | [2] | | |
| SMA | 1:1 | 40 | 20 | 2:1 | 1.6 | | PEA | 4.4 mg | 7, Clear | NS |
| | | | | | | | | [0.25] | | |
| | | | | | 1.64 | | PEA | Further | 10, Clear | |
| | | | | | | | | 2.9 mg. | | |
| | | | | | | | | 7.3 mg | | |
| | | | | | | | | [0.45] | | |
| | | | | | | | | (total) | | |
| SMA | 1:1 | 50 | 20 | 2.5:1 | 2 | 7 | Isopentylamine | 3 mg | 7, Clear | NS |
| | | | | | | | | [0.15] | | |
| SMA | 1:1 | 50 | 20 | 2.5:1 | 2 | | Isobutylamine | 9 mg | 8, clears | S |
| | | | | | | | | [0.45] | on | |
| | | | | | | | | | standing | |
| | | | | | | | | | 3 days | |
| SMA | 1:1 | 50 | 20 | 2.5:1 | 2 | 8 | Tyramine | 5-6 mg | 6&8 | S |
| | | | | | | | | [0.25-0.3] | clears | |
| SMA | 1:1 | 50 | 20 | 2.5:1 | 2 | 8 | Diphenhydramine | 2.5-4 mg | 7-8 | S |
| | | | | | | | | [0.13-0.2] | clears, | |
| | | | | | | | | | ppt 6 | |
| SMA | 1:1 | 50 | 20 | 2.5:1 | 2 | 8 | Phenylalanine | 10 mg | 8, Clears | Cleared, |
| | | | | | | | ethyl ester | [0.5] | | PS |
| SMA | 1:1 | 50 | 20 | 2.5:1 | 2 | 8 | 2-Benzyl-2- | 5 mg | 7-8, | Remains |
| | | | | | | | imidazoline | [0.25] | Clears | clear |
| | | | | | | | (tolazoline) | | | overnight, |
| | | | | | | | | | | S |
| SMA | 1:1 | 50 | 20 | 2.5:1 | 2 | 7 | Naphazoline | 5 mg, | 8, Clear | Virtually |
| | | | | | | | | Further 3 mg. | Cloudy 7, | clear |
| | | | | | | | | 8 mg | Clear 8-9. | PS |
| | | | | | | | | [0.4] | | |
| | | | | | | | | (total) | | |

Particle Size Studies

| | | | | | | | | | | |
|---|---|---|---|---|---|---|---|---|---|---|
| SMA | 1:1 | 50 | 20 | 2.5:1 | 2 | 8 | PEA | 8 mg | 8, clear. | Diam = |
| Test | Particle | | | | | | | [0.4] | Filtered | 54.2 nm |
| | size | | | | | | | | 0.2 μm. | |
| SMA | 1:1 | 50 | 20 | 2.5:1 | 2 | 5 | | | 4-5, | Diam = |
| Control | Particle | | | | | | | | Clear. | 93.5 nm |
| | size | | | | | | | | Filtered | |
| | | | | | | | | | 0.2 μm. | |

Poly Cation Example

| | | | | | | | | |
|---|---|---|---|---|---|---|---|---|
| Poly(itaconic | — | 50 | — | 2.5:1 | 1.5 | Benzoic acid | 26 mg | 5, Clear |
| Acid - | | | | | | | [1.7] | |
| Zinor) - | | | | | | | | |
| polycationic | | | | | | | | |
| (PIA-Zn) | | | | | | | | |

Poly Anion Example

| | | | | | | | | | | |
|---|---|---|---|---|---|---|---|---|---|---|
| Poly(methacrylic | | 50 | 10 | 5:1 | 2 | 6 | Isobutyl | 10 mg | 4, ppt | Clear |
| acid) | | | | | | | amine | [0.5] | | |
| sodium | | | | | | | | | | |
| 4-6k (S-A) | | | | | | | | | | |
| | | | | | | | Isobutyl | Further | 4, white | Clear |
| | | | | | | | amine | 14 mg, 24 mg, | ppt, 6 | overnight |
| | | | | | | | | [1.2] (total). | ppt, 5 | few white |
| | | | | | | | | Further | clear | ppt |
| | | | | | | | | 12 mg, 36 mg, | 7→9, | particles |
| | | | | | | | | [1.8] (total). | clouding | |
| | | | | | | | | | 5→3 | |
| | | | | | | | | | clear | |
| | | | | | | | | | 5, slight | |
| | | | | | | | | | clouding | |
| Poly(methacrylic | Preformed | 50 | 10 | 5:1 | 2 | 7-8 | 2-Benzyl-2- | 22 mg | 6, ppt & | |
| acid) | PMA + Bz | | | | | | imidazoline | [1.1] | clear, | |
| Sodium | | | | | | | (tolazoline) | | high | |
| 4-6k (S-A) | | | | | | | | | surface | |
| | | | | | | | | | tension | |
| | | | | | | | | | 5-2 | |

| | | | | | | | | |
|---|---|---|---|---|---|---|---|---|
| Poly(maleic acid) | | | | | | | | |
| ex PolyS | | | | | | | | |
| | 50 | | | 2 | 8 | Isobutyl | 15 mg | 7 |
| | | | | | | amine | [0.75] | |
| | | 2 | 25:1 | | | | Further 5 mg, | No |
| | | | | | | | 20 mg | clearing. |
| | | | | | | | [1.0] | |
| | | | | | | | (total) | |
| | | | | | | | Further 5 mg, | 8, No |
| | | | | | | | 25 mg | clearing. |
| | | | | | | | [1.25] | |
| | | | | | | | (total). | |
| | | Further | 12.5:1 | | | | | 3, Complete |
| | | 2. total = | | | | | | clearing |
| | | 4. | | | | | | fine |
| | | | | | | | | white |
| | | | | | | | | flecks, |
| | | | | | | | | 4 ppt. |

*Stability: S-stable, PS—partially stable, NS—not stable.

Specific Species Comprising a Cationic Counter Ion that Form Working Ion Pairs: Hydrochloride Salts Used of the Free Bases Shown Below:

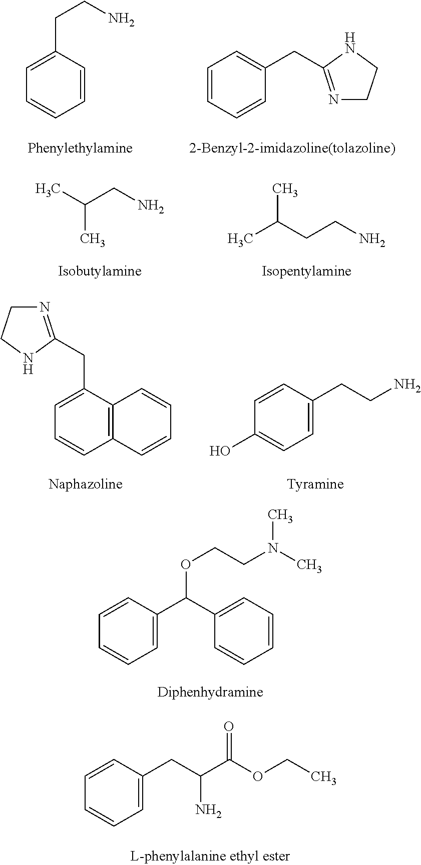

Specific Species Comprising a Counter Ion that Form Partial Working Ion Pairs: Hydrochloride Salts Used of Free the Base Shown Below: Forming Translucent Rather than Transparent Aqueous Solutions of Limited Stability.

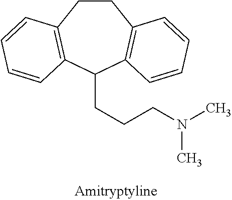 [SMILES: CN(C)CCCC1c2ccccc2CCc2ccccc21]

Specific Species Comprising a Counter Ion that Fail to Form Working Ion Pairs: Hydrochloride Salts Used of the Free Bases Shown Below:

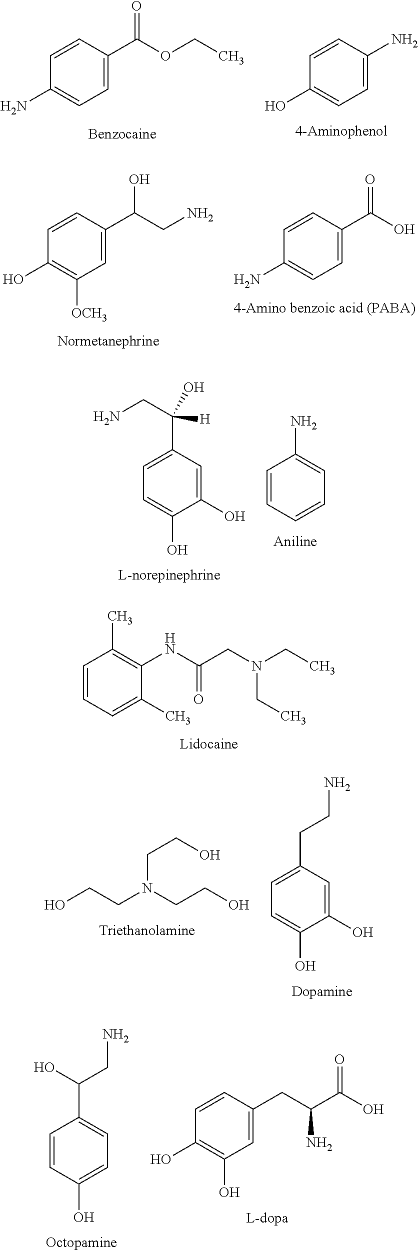

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps. As used herein, the term ‘between’ includes the recited end values.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1.-41. (canceled)

42. A composition comprising a lipid, a hydrophobically associating charged polymer and a species comprising a counter ion, wherein the counter ion is oppositely charged to the polymer and wherein the species comprising a counter ion further comprises a hydrophobic group.

43. The composition of claim 42 wherein the species comprising a counter ion is organic.

44. The composition of claim 42 wherein the charged polymer is anionic and the counter ion is cationic.

45. The composition of claim 44 wherein the counter ion is provided by a basic group.

46. The composition of claim 45 wherein the basic group is a functional group comprising nitrogen.

47. The composition of claim 46 wherein the functional group is selected from one or more of an amine, an imidazoline, an imine, an imide, an azide, an azo compound, or salts thereof.

48. The composition of claim 42 wherein the charged polymer comprises carboxylic acid groups.

49. The composition of claim 42 wherein the charged polymer is a copolymer of styrene and maleic acid.

50. The composition of claim 49 wherein the ratio of styrene to maleic acid monomer units is 1:1 to 5:1.

51. The composition of claim 42 wherein the charged polymer is cationic and the counter ion is anionic.

52. The composition of claim 51 wherein the counter ion is provided by an acidic group.

53. The composition of claim 42 wherein the hydrophobic group comprises an aryl group.

54. The composition of claim 42 wherein the species comprising a counter ion is phenethylamine (PEA) in its salt form.

55. The composition of claim 42 wherein the species comprising a counter ion is present at a mass of between about 50-250 Da.

56. The composition of claim 42 wherein the ratio of hydrophobically associating charged polymer to counter ion is 1:1 to 20:1.

57. The composition of claim 42 wherein the ratio of charged polymer to counter ion is 1:1 to 10:1.

58. The composition of claim 42, wherein the composition is stable in aqueous solution at a pH between 7-8.5.

59. The composition of claim 42 wherein the lipid is a phospholipid.

60. The composition of claim 42, wherein the ratio of charged polymer to lipid is greater than 1:2 on a weight basis.

61. The composition of claim 42 which further comprises an active agent.
